(19) United States
(12) Patent Application Publication
(10) Pub. No.: US 2025/0292183 A1
(43) Pub. Date: Sep. 18, 2025

(54) MANAGEMENT SYSTEM AND MANAGEMENT METHOD

(71) Applicant: Hitachi, Ltd., Tokyo (JP)

(72) Inventors: Takayuki NOZAKI, Tokyo (JP); Tomoakira KAWAI, Tokyo (JP)

(73) Assignee: Hitachi, Ltd., Tokyo (JP)

(21) Appl. No.: 18/999,020

(22) Filed: Dec. 23, 2024

(30) Foreign Application Priority Data
Mar. 14, 2024 (JP) 2024-040561

(51) Int. Cl. G06Q 10/0639; G06Q 50/04; G16B 5/00

(52) U.S. Cl. CPC G06Q 10/06395; G06Q 50/04; G16B 5/00

(57) ABSTRACT

A management system includes an arithmetic operation section for simulating a culture process based on parameters of processing of culture target cells by culture and cell attribute information regarding the probability distribution of cell attributes of the cells, and an output section outputting a first manufacturing parameter classification criterion based on culture experiment data of the cells and quality criterion related to the cells, the information about the first manufacturing criterion comprising manufacturing conditions that are satisfied when the cells are cultured. The arithmetic operation section calculates evaluation indicator information based on results of the simulation and the quality criterion, the evaluation indicator information being an evaluation indicator regarding a quality indicator at the time when the cells are cultured using each of values included in the parameter information, and causes information about a revision proposal for the first manufacturing parameter classification criterion based on the evaluation indicator information.

### CROSS-REFERENCE TO RELATED APPLICATIONS

The present invention relates to and asserts priority from Japanese patent application No. 2024-040561 filed on Mar. 14, 2024, and incorporates the entirety of the contents and subject matter of all the above application herein by reference.

### BACKGROUND OF THE INVENTION

### Field of the Invention

The present invention relates to a management system and a management method.

### Description of the Related Art

Regenerative medicine is medicine to use regenerated tissues or cells to restore tissues or the like that are experiencing malfunctions or damage which is difficult to treat with conventional methods. In a series of processes until treatment, a biological sample is harvested from a patient her/himself or from another person at a medical institution, for example. After the harvesting, the biological sample is transported to a cell processing facility (CPF). At the CPF, separation, purification, gene transfer, and the like are performed on the biological sample to grow and organize cells by culture or the like, and a cell-processed product satisfying a quality evaluation criterion is transported to a medical institution or the like where the cell-processed product is used for treatment of the patient.

Conventionally, the quality of manufacturing of a cell-processed product in regenerative medicine has been guaranteed by results of a post-manufacturing final quality test on the basis of the concept of quality by test (QbT), as with pharmaceuticals. Recently, the introduction of quality by design (QbD) in which quality is built in the course of product development has been underway for pharmaceuticals. In QbD, quality is secured by understanding a product and its development processes and manufacturing processes, and managing and constructing production processes of the product. In QbD, a quality target product profile (QTPP) of a product under development is clarified, quality attributes (QAs) required for the product with regard to the QTPP are extracted, and a critical quality attribute (CQA) which is a particularly important quality attribute for quality assurance is specified. In QbD, a critical material attribute (CMA) and a critical process parameter (CPP) are specified from material attributes (MAs) and process parameters (PPs) that influence the CQA, and a management strategy is constructed. Further, in QbD, a design space where production in any scale or lot is possible is also set. The design space is an area formed by a multi-dimensional combination of input variables (a CPP, etc.) such as parameters of manufacturing steps, and interactions, and, if a manufactured cell-processed product is positioned in this area, this means that it is proven that the quality is secured. In addition, it is also considered effective to determine a tolerance range of parameters that influence quality, and set a manufacturing parameter classification criterion representing parameters in the tolerance range. If a cell-processed product is manufactured with the parameters in the tolerance range, the quality of the cell-processed product satisfies a shipment classification criterion. The suitability of a management strategy is examined continuously, and the management strategy is improved. Note that, in contrast to a design space that specifies a range by mainly using important factors, a manufacturing parameter classification criterion specifies a range within a tolerance range of parameters which influence quality and which can be set as desired (it does not matter whether or not the parameters are important factors). Hence, the manufacturing parameter classification criterion can be said to be a concept which is an expansion of the design space or, in other words, a concept encompassing the design space. Accordingly, the concept of the manufacturing parameter classification criterion includes the design space based on QbD, and also includes a CQA, a CMA, a CPP, and the like for specifying the design space. In the following explanation, unless it is necessary to make a distinction between a manufacturing parameter classification criterion and a design space, the manufacturing parameter classification criterion and the design space are collectively called a “manufacturing parameter classification criterion” simply.

Although the introduction of QbD has been underway also for regenerative medicine, there are differences that it is difficult to standardize quality as compared to pharmaceuticals because cells or a biological sample are/is handled as a raw material in regenerative medicine, that there are many steps to be performed by manual work currently, which can cause variations in work contents, and so on. Therefore, in the introduction of QbD to regenerative medicine, it is necessary to accumulate and manage various types of information in the life cycle of a cell-processed product or the like, like information about respective steps such as harvesting, purification, gene transfer, culture, condensation, transportation, or transplanting, information about material management of raw materials and the like, or medical treatment information such as post-transplantation adverse events and/or safety information, to understand relations and the like among quality attributes, variation attributes, treatment outcomes, and the like, to find indicators and the like that influence the quality, and to construct a management strategy.

However, as mentioned before, in regenerative medicine, cells or a biological sample whose quality is difficult to standardize is used as a raw material, and besides, there are currently many steps to be performed by manual work. Cells themselves vary at steps such as culture. That is, disturbance occurs to cell attributes. In addition, in the case of steps to be performed by manual work, there can be variations among operators even if the same parameters are used. In the case of automated steps, too, an apparatus itself can generate variations caused by control errors or the like. In constructing a management strategy on the basis of QbD, as with pharmaceuticals, it is necessary to determine parameters through experiments such that the quality of a cell-processed product is positioned in a manufacturing parameter classification criterion. However, experiments using cells require cost and time, and in addition, it is also difficult to secure experiment reproducibility due to the disturbance of cell attributes.

JP-T-2023-544029 discloses a method targeted at cells manufactured by tank culture. The method is for improving a post-manufacturing yield increase or the like, and includes, for each of one or more time intervals during a cell culture process obtaining current values of one or more cell culture attributes associated with a cell culture, predicting one or more future values of a particular cell culture attribute associated with the cell culture, and controlling one or more physical inputs to the cell culture process.

In addition, WO 2023-276450 discloses a method targeted at cells. The method is for predicting a cell culture result, and includes receiving an input of a culture environment including a culture medium composition and a culture condition for culturing cells, predicting a biological behavior amount on the basis of the culture environment, changing a cell environment on the basis of the predicted biological behavior amount, repeating prediction of the biological behavior amount and change of the cell environment on the basis of the changed cell environment, and outputting the changed cell environment.

### SUMMARY OF THE INVENTION

In the methods disclosed in JP-T-2023-544029 and WO 2023-276450, a manufacturing parameter classification criterion (e.g., a design space) is fixed, manufacturing with each parameter is evaluated by a simulation, and parameters that bring about better manufacturing results can be determined. However, neither the method disclosed in JP-T-2023-544029 nor the method disclosed in WO 2023-276450 performs evaluation of a manufacturing parameter classification criterion aimed for quality assurance. As mentioned before, in regenerative medicine, cells or biological samples are handled as a raw material, there are many steps to be performed by manual work currently, experiments for deciding a manufacturing parameter classification criterion require cost and time, and it is also difficult to secure experiment reproducibility due to the disturbance of cell attributes. Therefore, it is not easy to increase the number of times of experiments and to obtain reliable data highly accurately. Accordingly, a manufacturing parameter classification criterion may be evaluated by a method other than experiments, but neither JP-T-2023-544029 nor WO 2023-276450 mentions such a method. In addition, a manufacturing parameter classification criterion, in particular, a design space, is decided through experiments on the basis of QbD, but, for that purpose, it is desirable that many conditions be changed, experiments be implemented a large number of times, and reliable data be obtained highly accurately. However, these are difficult in reality due to the circumstances described before.

Due to the circumstances explained above, it has been difficult to realize quality stabilization of a cell-processed product with conventional technologies.

The present invention has been made to solve problems like this. An object of the present invention is to provide a management system and a management method that can realize quality stabilization of a cell-processed product.

A management system according to the present invention having solved the problem includes a storage section that stores cell attribute information which is information about a probability distribution of a cell attribute of culture target cells, experiment data of an experiment of culturing the culture target cells, and information about a quality criterion of a product related to the culture target cells, an input section to which parameter information is input, the parameter information being related to a parameter of processing of the culture target cells by at least culture, an arithmetic operation section that performs a simulation of a culture process of the culture target cells on the basis of the parameter information input to the input section and the cell attribute information, and an output section that outputs information about a first manufacturing parameter classification criterion on the basis of the experiment data and the information about the quality criterion, the information about the first manufacturing parameter classification criterion being information about a manufacturing condition that should be satisfied when the culture target cells are cultured. The arithmetic operation section is configured to calculate evaluation indicator information on the basis of a result of the simulation and the information about the quality criterion, the evaluation indicator information being an evaluation indicator with regard to the quality criterion of a quality indicator at the time when the culture target cells are cultured using each of values included in the parameter information, and cause information about a revision proposal for the first manufacturing parameter classification criterion based on the evaluation indicator information to be output.

The present invention can provide a management system and a management method that can realize quality stabilization of a cell-processed product.

Problems, configurations, and effects other than those mentioned before are made clear by the following explanation of embodiments.

### BRIEF DESCRIPTION OF THE DRAWINGS

FIG. 1 is a configuration diagram depicting a configuration of a management system according to a first embodiment;

FIG. 2A is a block diagram depicting occurrence of all pieces of information in a life cycle of a cell-processed product or the like accumulated and/or managed by the management system;

FIG. 2B is an explanatory diagram depicting examples of quality information and manufacturing information;

FIG. 3 is an explanatory diagram depicting a screen example of the management system according to the first embodiment;

FIG. 4A depicts an example of a graph of experiment values of each parameter and a quality indicator, and is a graph depicting an example of results obtained by changing one parameter and plotting one quality indicator;

FIG. 4B depicts an example of a graph of experiment values of each parameter and a quality indicator, and is a graph depicting an example of results obtained by changing two parameters and plotting one quality indicator;

FIG. 4C is an explanatory diagram depicting a screen example of a table (the figure on the left) of each input variable included in a manufacturing parameter classification criterion and values of each quality indicator obtained when parameters are changed, and a graph (the figure on the right) based on the table;

FIG. 5A is an explanatory diagram depicting an example of a two-dimensional manufacturing parameter classification criterion;

FIG. 5B is an explanatory diagram depicting an example of a three-dimensional manufacturing parameter classification criterion;

FIG. 5C is an explanatory diagram depicting an example of a one-dimensional manufacturing parameter classification criterion;

FIG. 5D is an explanatory diagram depicting a screen example of a table (the figure on the left) of each input variable included in a manufacturing parameter classification criterion and values of each quality indicator obtained when parameters are changed, and a graph (the figure on the right) on which lots (Nos. 1 to 17) at the time when each parameter in the manufacturing parameter classification criterion is changed are plotted;

FIG. 6 is an explanatory diagram depicting an example of computation results obtained by performing a cell culture simulation taking into consideration cellular fluctuations;

FIG. 7A is an explanatory diagram depicting a screen example of a table (the figure on the upper left) depicting computation results obtained by performing a cell culture simulation taking into consideration cellular fluctuations with regard to a manufacturing parameter classification criterion determined through experiments, and graphs (the figure on the upper right, the figure on the lower left, and the figure on the lower right) on which the computation results are plotted;

FIG. 7B is a graph depicting an example of results obtained by changing one parameter and plotting one quality indicator;

FIG. 7C is a graph depicting another example of results obtained by changing one parameter and plotting one quality indicator;

FIG. 8A is an explanatory diagram for explaining an indicator for evaluating the degree of stability of computation results in the case of manufacturing with each parameter;

FIG. 8B is an explanatory diagram for explaining another indicator for evaluating the degree of stability of computation results in the case of manufacturing with each parameter;

FIG. 8C is an explanatory diagram for explaining another indicator for evaluating the degree of stability of computation results in the case of manufacturing with each parameter;

FIG. 9A is an explanatory diagram depicting a screen example presenting a revision proposal for a manufacturing parameter classification criterion created on the basis of experiment data, by using scores and ranks;

FIG. 9B is an explanatory diagram depicting another screen example presenting a revision proposal for a manufacturing parameter classification criterion created on the basis of experiment data, by using scores and ranks;

FIG. 10A is an explanatory diagram depicting a screen example of the management system used by a management user;

FIG. 10B is an explanatory diagram depicting a screen example of the management system used by a management user;

FIG. 10C is an explanatory diagram depicting a screen example of the management system used by a management user;

FIG. 11 is a flowchart for explaining contents of a management method according to the first embodiment;

FIG. 12 is a flowchart for explaining a series of more specific procedures of the management method according to the first embodiment; and

FIG. 13 is a flowchart for explaining a series of specific procedures of a management method according to a fourth embodiment.

### DESCRIPTION OF THE PREFERRED EMBODIMENTS

In order to achieve the object described above, the present invention has the following configuration. Note that objects, features, and advantages of the present invention as well as ideas thereof are apparent to those skilled in the art from the description of the present specification, and those skilled in the art can easily reproduce the present invention from the description of the present specification. Specific embodiments or the like of the invention described below represent preferred embodiments of the present invention, are depicted for illustration or explanation, and do not limit the present invention to them. It is apparent to those skilled in the art that various alterations and modifications can be made on the basis of the description of the present specification within the intension and scope of the present invention disclosed in the present specification.

The following explains one embodiment according to the present invention with reference to the figures. Embodiments explained below illustrate examples for explaining the present invention, and there are omissions and simplifications as appropriate for clarification of the explanation. The present invention can be implemented also in various other modes. Unless there is a particular limitation, the number of each constituent element may be one or greater than one. The position, size, shape, range, and the like of each constituent element depicted in the figures do not represent the actual position, size, shape, range, and the like in some cases, for making it easy to understand the invention. Accordingly, the present invention is not necessarily limited by positions, sizes, shapes, ranges, and the like disclosed in the figures.

In a case where there are a plurality of constituent elements having identical or similar functions, they are explained by being given different suffixes to identical reference characters in some cases. In addition, unless it is necessary to make distinctions between the plurality of constituent elements, they are explained without the suffixes in some cases. Overlapping explanations of constituent elements having identical or similar functions are omitted in some cases.

In embodiments, processes to be performed by execution of a program are explained in some cases. Here, a computer executes a program by using a processor (e.g., a central processing unit (CPU), a graphics processing unit (GPU)), and performs processes specified in the program, by using a storage resource (e.g., a memory), an interface device (e.g., a communication port), and the like. Accordingly, the subject of processes to be performed by execution of a program may be the processor. Similarly, the subject of processes to be performed by execution of a program may be a controller, an apparatus, a system, a computer, or a node having the processor. It is sufficient if the subject of processes to be performed by execution of a program is an arithmetic operation section, and may include a dedicated circuit that performs particular processes. Here, for example, the dedicated circuit is a field programmable gate array (FPGA), an application specific integrated circuit (ASIC), a complex programmable logic device (CPLD), or the like.

A program may be installed on a computer from a program source. For example, the program source may be a program distribution server or a computer-readable storage medium. In a case where the program source is a program distribution server, the program distribution server may include a processor and a storage resource that stores a distribution target program, and the processor of the program distribution server may distribute the distribution target program to another computer. In addition, in embodiments, two or more programs may be realized as one program, or one program may be realized as two or more programs.

The present invention relates to a management system and a management method that process parameters associated with a cell-processed product. In the present invention, for example, regarding a cell-processed product, the influence of each parameter on the quality is evaluated by a cell culture simulation taking into consideration cellular fluctuations with regard to a manufacturing parameter classification criterion (including a design space) created on the basis of experiment data, and the manufacturing parameter classification criterion (first manufacturing parameter classification criterion) created on the basis of the experiment data is revised. Note that the breadth and narrowness of concepts of manufacturing parameter classification criteria and design spaces are as mentioned before. It is assumed in the following explanation that the terms “manufacturing parameter classification criterion” and “design space” are interchangeable. In addition, the term “design space” is used for explanation that is intended to mention a design space in particular.

### First Embodiment

### <Management System>

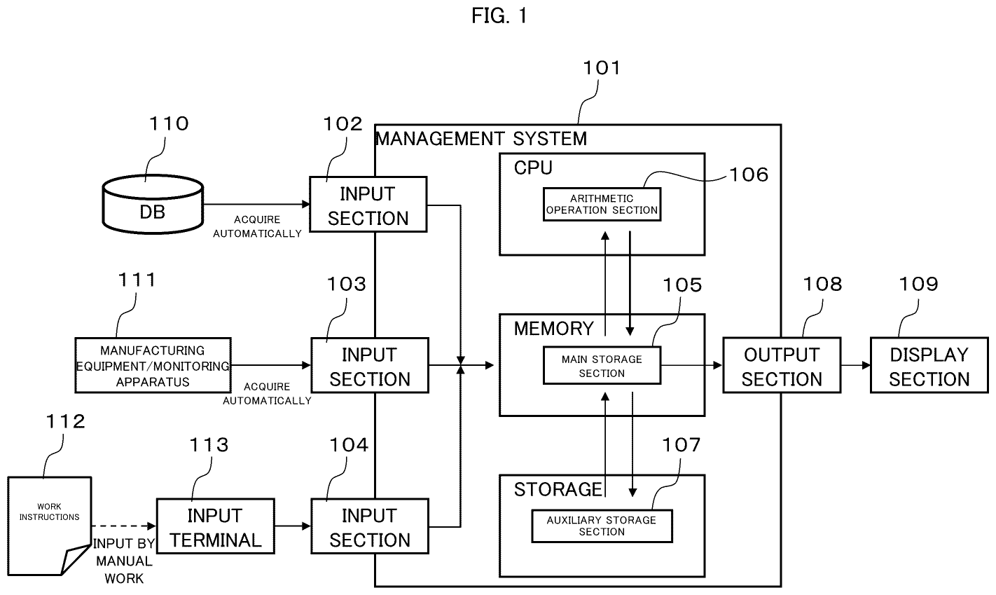

FIG. 1 is a configuration diagram depicting a configuration of a management system 101 according to a first embodiment. The management system 101 includes input sections 102, 103, and 104 (input apparatuses), an output section 108 (output apparatus), an arithmetic operation section 106 (processor), a main storage section 105 (storage apparatus), and an auxiliary storage section 107 (storage apparatus).

The management system 101 handles information about a cell-processed product. In the present specification, cell-processed products widely encompass products manufactured using cells or cell tissues, and in particular, include products related to regenerative medicine or the like specified in laws (Act on Securing Quality, Efficacy and Safety of Products Including Pharmaceuticals and Medical Devices) related to securing and the like of the quality, efficacy, and safety of medical devices or the like, and particular cell-processed products specified in laws (Act on Securing Safety of Regenerative Medicine) related to securing and the like of the safety of regenerative medicine or the like. Units of cell-processed products can be defined as desired, and for example, each manufacturing lot can be defined as one unit of a cell-processed product.

The input sections 102, 103, and 104 have mechanisms to import various types of data by being coordinated with an external system or an equipped device or by supporting manual input of data. They import, as an input, each parameter related to manufacturing or the like, and may be three types of input section supporting different import methods. That is, through the input sections 102, 103, and 104, parameter information related to parameters of processing of culture target cells by at least culture is input.

Data and parameters imported through the input sections 102, 103, and 104 are temporarily stored on the main storage section 105 in a memory. Then, the acquired data is processed and/or concatenated at the arithmetic operation section 106 in a CPU, and valorized. For example, the arithmetic operation section 106 determines a manufacturing parameter classification criterion from experiment values which are an input, and the arithmetic operation section 106 further performs a simulation of a culture process from each parameter. Then, the arithmetic operation section 106 computes quality information corresponding to each parameter, and compares the manufacturing parameter classification criterion determined from the experiment values with the quality information corresponding to each parameter determined by the computation. Then, the arithmetic operation section 106 causes information about a revision proposal for the manufacturing parameter classification criterion determined from the experiment values to be output. That is, on the basis of results of the simulation described before and information about a quality criterion, for each of values included in the parameter information described before, the arithmetic operation section 106 calculates evaluation indicator information (i.e., the quality information described before) which is an evaluation indicator with regard to a quality criterion of a quality indicator at the time when culture target cells are cultured using the value, and causes the output section 108 to output information about a revision proposal for a first manufacturing parameter classification criterion based on the evaluation indicator information. Note that, for example, the parameters (manufacturing parameters) are various factors at the time of manufacturing and at the time of culture, such as serum lots, flow rates, discharge positions, lengths of work time, or work-time temperatures, and are not limited to those described before. It is sufficient if the parameter information is information about parameters. Specific examples include serum lot numbers, values of flow rates, coordinates of discharge positions, lengths of time required for work, temperatures of a room at the time of work, and the like. For example, examples of quality indicators include a cell count, cell viability, purity, and the like. Anything can be an quality indicator as long as it is information about tissues or cells obtained by culture, and the quality indicators are not limited to those described before. The number of quality indicators is greater than one in many cases. The evaluation indicator information is set as appropriate depending on a quality indicator. In addition, the number of manufacturing parameter classification criteria can be greater than one. A manufacturing parameter classification criterion which is one selected from a plurality of manufacturing parameter classification criteria or which is the only one manufacturing parameter classification criterion is called a “first manufacturing parameter classification criterion” in some cases.

Results therefrom and imported original data are stored on the auxiliary storage section 107 in a storage. They are output to an outside of the management system 101 via the output section 108, and the data is displayed on a display section 109. In a modification example, the display section 109 may be configured as an output apparatus of the management system 101. For example, examples of the display section 109 include a display, a printer, a speaker, and the like. Examples of the output section 108 include a high-definition multimedia interface (HDMI) terminal, a DisplayPort terminal, a Mini DisplayPort terminal, a digital visual interface (DVI) terminal, a universal serial bus (USB) Type-C terminal, a video graphics array (VGA) terminal, and the like.

The main storage section 105 and/or the auxiliary storage section 107 are equivalent to what is generally called a storage section. The main storage section 105 and/or the auxiliary storage section 107 may store a program. By execution of the program by the arithmetic operation section 106, the management system 101, which is a computer, may execute functions explained in the present embodiment. That is, the program causes the computer to function as the management system 101 according to the present embodiment. For example, the main storage section 105 and/or the auxiliary storage section 107 may be a read only memory (ROM), a random access memory (RAM), or a hard disk drive (HDD), instead of a memory.

In addition, the main storage section 105 and/or the auxiliary storage section 107 may store cell attribute information which is information about a probability distribution of cell attributes, experiment data obtained through experiments, information about a quality criterion of a product, and the like. Note that part or the whole of these pieces of information and the experiment data may be stored on a storage apparatus or a storage system outside the configuration of the management system 101.

The input sections 102, 103, and 104 have different functions corresponding to mutually different data import methods. The input section 102 imports data by being coordinated with an external system. Examples of the external system include a manufacturing execution system (MES), a material management system, an electronic medical record, a patient registry, a laboratory information management system (LIMS), and the like. The input section 102 accesses a database (DB) 110 of each system and imports data that it has referred to.

The input section 103 imports data by being coordinated with manufacturing equipment, a monitoring apparatus, and the like. Examples of the manufacturing equipment and the monitoring apparatus include an automatic culture apparatus that automatically cultures cells, a cell observation system, and the like. In addition, examples of the manufacturing equipment and the monitoring apparatus include a cleanliness monitoring apparatus that monitors the number of floating fungi or the number of particles in a manufacturing environment, and the like. In addition, examples of the manufacturing equipment and the monitoring apparatus include a monitoring system that monitors contents of work implemented by manual work, motions of an operator at manufacturing equipment, and the like, a transportation monitoring apparatus that measures temperature and/or pressure at the time of transportation, and the like.

The input section 103 accesses a manufacturing equipment/monitoring apparatus 111 (at least either the manufacturing equipment or the monitoring apparatus) like these, and imports data. Note that, in a case where data obtained by the manufacturing equipment/monitoring apparatus 111 is stored temporarily on some system and the database 110 is referred to, the input section 102 is used. It is assumed that the input section 103 is used in a case where data is imported directly from the manufacturing equipment/monitoring apparatus 111 bypassing the database 110.

The input section 104 imports manually input data. For example, in regenerative medicine, there can be also a step implemented by an operator by manual work in accordance with work instructions 112. In that case in particular, there can be cases where results of work implemented by manual work or monitoring results obtained by measurement during work are written in the work instructions 112 by manual work. The input section 104 supports a data import method assuming that, for example, after an operator or the like has performed work, the operator inputs contents described in the work instructions 112, via an input terminal 113 by manual work. In addition, data may be imported directly from the work instructions 112 via the input section 103. For example, the input section 104 may be a keyboard, a mouse, a touch panel, a numeric keypad, a scanner, a microphone, or a sensor.

Note that work results or monitoring results obtained by measurement during work are recorded on an electronic terminal in some cases. In that case, there can also be cases where the input section 103 directly imports data or cases where data is temporarily stored on the database 110 and is imported through the input section 102. Note that data to be imported is not limited to data that occurs at the time of commercial production after the acquisition of a manufacture and sales approval, for example, and information that occurs over the entire life cycle of a cell-processed product or the like during a clinical trial, a clinical research, a basic research, and the like may be collected. It is considered that, as the information amount increases, the precision of analysis also rises by a corresponding amount. It should be noted that, in that case, it is also quite possible that the type, amount, and/or quality of information differs depending on the timing of development, and it is preferable to handle data taking such differences into consideration.

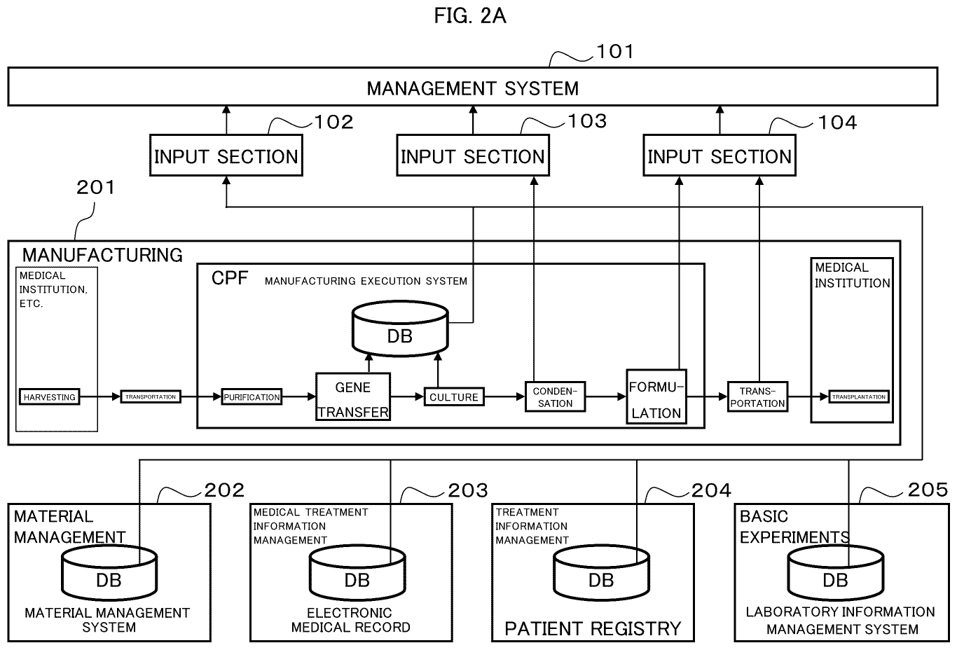

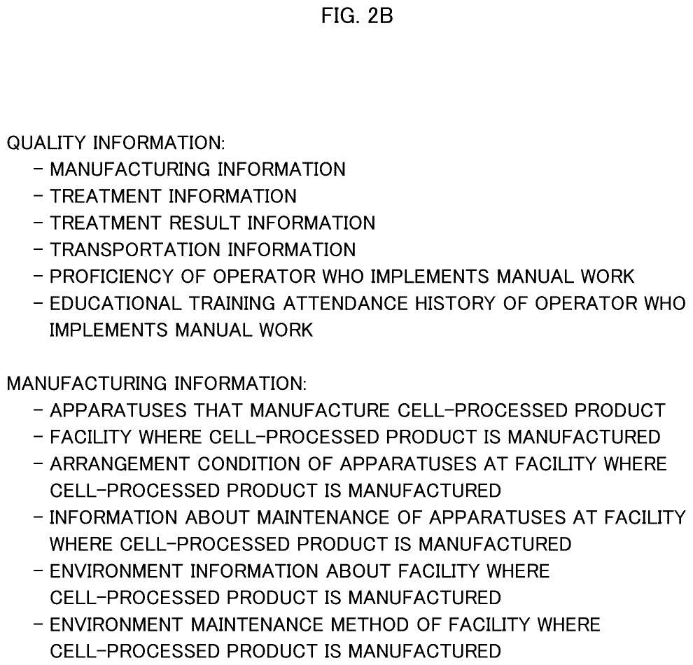

FIG. 2A is a block diagram depicting occurrence of all pieces of information in the life cycle of a cell-processed product or the like accumulated and/or managed by the management system 101. FIG. 2A depicts an example of occurrence of various types of information (quality information) in the life cycle of a cell-processed product or the like, like data related to manufacturing of the cell-processed product or the like accumulated and/or managed by the management system 101, material management of raw materials and the like, medical treatment information management, treatment information management of post-transplantation adverse events and/or safety information, or basic experiments.

As depicted in FIG. 2A, each step related to manufacturing 201 can change depending on the type of a cell-processed product or the like to be manufactured and the type of a treatment target disorder. Here, harvesting, purification, gene transfer, culture, condensation, formulation, and transplantation steps are depicted. A transportation step may be included. In addition, although it is assumed that pieces of data related to material management 202, medical treatment information management 203, treatment information management 204, and basic experiments 205 are accumulated in a material management system, an electronic medical record, a patient registry, and a laboratory information management system, respectively, another method may be adopted.

Whereas the three types of input section, namely, the input sections 102 to 104, have been mentioned with reference to FIG. 1, in manufacturing of a cell-processed product or the like, locations of implementation can depend on steps as depicted in FIG. 2A. For example, the harvesting step can be performed at a medical institution or the like. Purification, gene transfer, culture, condensation, and formulation are typically performed at a CPF. Types of input section to be used, the input sections 102 to 104, can depend on locations of implementation. In addition, even for steps or contents of work implemented in the same CPF, types of input section to be used, the input sections 102 to 104, may vary for each of the steps or for each of more subdivided work contents.

For example, in a case where an automatic culture apparatus is used at the culture step, data may be input directly to the management system 101 through the input section 103 depicted in FIG. 1, or data may be temporarily input to the database 110 of a manufacturing execution system and then input to the management system 101 through the input section 102. Alternatively, in a case where an operator implements the culture step by manual work in a safety cabinet and writes work results or the like in the work instructions 112 by manual work, there can be cases where the operator manually inputs data by using the input section 104. Further, in a case where manufacturing, transplantation, and the like are implemented at a plurality of facilities, work contents or data occurrence methods can vary also for each facility, and accordingly, input schemes to be used also can vary. Note that, as an example, FIG. 2A depicts a case where the input section 102 is used for the gene transfer and culture steps, the input section 103 is used for the condensation step, and the input section 104 is used for the formulation and transportation steps.

Various types of information in the life cycle of a cell-processed product or the like accumulated and/or managed by the management system 101 are explained. FIG. 2B is an explanatory diagram for explaining an example of various types of information in the life cycle of a cell-processed product or the like accumulated and/or managed by the management system 101. As depicted in FIG. 2B, for example, the various types of information include quality information and manufacturing information.

The quality information includes a plurality of parameters related to a plurality of cell-processed products, and in particular, includes parameters related to at least one of manufacturing information, treatment information, treatment result information, and transportation information related to the cell-processed products.

In addition, the quality information may include proficiency of an operator who implements manual work and/or an educational training attendance history of an operator who implements manual work. By doing so, quality evaluation taking into consideration the skill of an operator becomes possible.

As depicted in FIG. 2B, the manufacturing information described before may include at least one of apparatuses that manufacture a cell-processed product, a facility where the cell-processed product is manufactured, the arrangement condition of apparatuses at the facility where the cell-processed product is manufactured, information about maintenance of the apparatuses at the facility where the cell-processed product is manufactured, environment information (e.g., cleanliness) about the facility where the cell-processed product is manufactured, and an environment maintenance method (e.g., a cleaning method) of the facility where the cell-processed product is manufactured. By doing so, various types of information about the cell-processed product can be handled.

Naturally, the manufacturing information may include information other than these. Examples of information other than these are explained below.

The manufacturing information described before is classified into information about implementation by manual work and information about implementation by automated manufacturing equipment, for each step or for each more subdivided work content. Depending on which of these types the information is, the type of data to occur and/or the number of pieces of the data can be different. Typically, the work contents of manual work are numerically unspecified, and also work results are not recorded in detail, as compared to work by automated manufacturing equipment. However, no matter whether work is implemented by manual work or by automated manufacturing equipment, by accumulating every possible piece of data, the accumulated data is made available for subsequent analysis.

For example, examples of the manufacturing information about cell seeding work at the culture step include a cell count at the time of harvesting of cells to be seeded, cell viability, an expression level of particular protein, a culture container to be used for culture, and the like. Accompanying information about the culture container to be used for culture also includes the manufacturer name, the lot number, the date of manufacturing, the use deadline, and the like in addition to the type (the shape, the type of a base material, a culture scheme), and these pieces of data may be accumulated in the material management system for material management.

In addition, for example, examples of the manufacturing information about cell seeding work at the culture step include the type of a solvent of a cell suspension at the time of seeding, the solvent amount, the solvent composition, the cell seeding density, the medium amount at the time of culture, the culture medium composition, the liquid feeding speed at the time of seeding, the liquid feeding location, the cell distribution in the culture container after seeding, shear stress having occurred to cells, the total length of work time, and the like.

Examples of the transportation information described before include the conveyance speed, vibrations at the time of conveyance, the total conveyance time, and the like in a case where the culture container is conveyed after seeding from a work location such as a safety cabinet to an incubator where culture is performed. The transportation information may include the temperature and/or pressure at the time of transportation.

These items can influence the post-manufacturing quality of a cell-processed product or the like or the quality of an intermediate product to a greater or lesser degree, and the degree of the influence and the manner of the influence are evaluated by the management system 101. For example, it can be expected that, in a case where the cell viability at the time of harvesting is low, the activity of cells at subsequent culture is low. In a case where the liquid feeding speed at the time of seeding is too fast, the magnitude of shear stress received by cells also becomes great, and there is a possibility that subsequent growth of the cells is influenced.

Typically, in cell seeding, the temperature and gas phase inside a safety cabinet or the like is often not controlled as compared to an incubator where culture is performed. Hence, a temperature reduction and/or a medium pH change occurs depending on the length of work time of seeding, and this can influence cells. In a case where cells are conveyed from a work location such as a safety cabinet to an incubator where culture is performed, vibrations at the time of conveyance give vibrations and/or shocks to the cells. It is possible that occurrence of a force to the cells and a change in the cell distribution in the culture container, depending on acceleration due to a conveyance speed change or depending on a route, influence culture results thereafter. These pieces of information may be included in the transportation information.

The total conveyance time can influence a temperature change and a medium pH change as the length of work time of seeding does. Note that, in many cases, the scope of control and items to be monitored differ between work performed by automated manufacturing equipment and work by manual work for reasons such as cost and/or efforts. It is quite possible that, in the future, the scope of control is expanded or items to be monitored are increased to increase types of data and/or the number of pieces of the data such that analysis by the management system 101 becomes more precise. These pieces of information may be included in the transportation information.

In particular, in a case where manufacturing and/or transplantation are/is performed at a plurality of facilities and differences between the facilities can influence the quality, information about the differences is also important for enabling analysis at the management system 101. Regarding devices being used, examples of the information include the manufacturer names, the model numbers, maintenance information (the dates of implementation, the frequency, contents of the maintenance), and methods of initialization to be performed every time the devices are used. These pieces of information may be included in the manufacturing information.

Regarding steps of manual work by operators, typically, proficiency and education histories are different among operators at different facilities. In addition, even if manual work is performed by the same operator, work contents and/or work results can differ every time the work is implemented. Therefore, information such as operator numbers, proficiency, or education histories associated with operator names is also important. Regarding the layout of a cell preparation room, information such as the number of apparatuses, the distances between the apparatuses, or routes is also important. These pieces of information may be included in the quality information.

For example, because a space where an operator conveys a culture container from the inside of a safety cabinet to an incubator is a space where temperature and/or a gas phase is not controlled, a temperature reduction and a pH change can influence cells. Therefore, for example, there is a possibility that the conveyance time influences the quality. Regarding the manufacturing environment, information such as the temperature or cleanliness of a cell preparation room or the like at a CPF, a use method related to aseptic manipulation or the like performed by an operator, a cleaning method and the cleaning frequency of a cell preparation room or the like, or an entrance/exit method such as gowning of an operator is also important. These pieces of information may be included in the quality information.

Examples of information associated with the harvesting step depicted in FIG. 2A include information about donors from whom tissues to be harvested are provided, and the like. Information about providers or the like is accumulated as donor information in the management system 101. Specific examples of the donor information include the registration dates of donors, the dates of acquisition of informed consent, registration identifications (IDs), the ages, the histories of provision of biological samples, the harvested-cell counts at the time of most recent provision, the medical histories of infections or the like, the heights, the body weights, the travel histories, results of blood tests and serological tests, and the like. These pieces of information may be included in the quality information.

Data that can be accumulated in the material management system for the material management 202 includes the names of manufacturers of used materials, the lot numbers, the dates of manufacturing, the use deadlines, types (the shapes and/or the types of base materials, etc.), and the like. It is assumed in the present embodiment that the data is information (material management information) to be managed when materials are purchased. These pieces of information may be included in the quality information.

Regarding the lot numbers, it is thought that, in particular, the lot numbers of serums to be used in culture significantly influence culture results.

For example, specific examples of the material management information include the order numbers, the orderers, the dates of orders, the item names, the makers, the items, the dates of delivery, the prices of purchases, information about the delivery destinations, the use deadlines, and the like. In the case of samples derived from living bodies, animal species, used portions, and use steps are added further. Because it is also expected that some materials are used as divided portions by dispensation or the like, information such as the dates of opening, dispensation containers, the number of dispensation containers, the dispensation volumes, or branch numbers also occurs, but these pieces of information may be handled as manufacturing information. This is because the information is also associated with information about the dates of work, operators, and work results in a case where work such as dispensation is performed. These pieces of information may be included in the quality information.

Data that can be accumulated in the electronic medical record for the medical treatment information management 203 includes basic information, medical treatment information, and the like about patients for whom transplantation is performed. Note that, because it is also expected that the data overlaps data that can be accumulated in the patient registry for the treatment information management mentioned later, in that case, it is preferable to adjust overlapping data. For example, specific examples of the medical treatment information include medical institution names, target-disorder patient IDs, the dates of consent acquisition, the dates of birth, the genders, the heights, the body weights, primary disorders, medical histories, complications, allergies, the dates of transplantation or the dates of start of transplantation, the dates of end of transplantation, and the like. These pieces of information may be included in the quality information.

Data that is included in the quality information and can be accumulated in the patient registry for the treatment information management 204 includes treatment information and treatment result information. Specific examples of the treatment information include information about cells having been transplanted, raw material information, manufacturing step procedure information, the dates and times of transplantation, doses, people who are in charge of administration, efficacy information (whether or not complete responses have been achieved at time points which are particular days after administration, survival statuses), and the like. Examples of the treatment result information include post-transplantation efficacy, adverse events, safety information, adverse event information (whether or not there is occurrence of adverse events, the total of all adverse events, the names of adverse events, event onset dates, seriousness, treatments and the like for adverse events, the dates of outcome, causal relation evaluation), and the like. As more detailed data, for example, the adverse event information may include infections/parasitic diseases, benign/malignant/unspecified neoplasms, blood/lymphatic system disorders, immune system disorders, endocrine system disorders, metabolic/nutritional disorders, mental disorders, nervous system disorders, eye disorders, ear/labyrinth disorders, cardiac disorders, vascular disorders, respiratory/thoracic/mediastinal disorders, gastrointestinal disorders, hepatobiliary system disorders, skin/subcutaneous tissue disorders, musculoskeletal/connective tissue disorders, renal/urinary tract disorders, reproductive system/breast disorders, congenital/familial/genetic disorders, and the like.

Examples of data that can be accumulated in the laboratory information management system for the basic experiments 205 include data as basic researches at an initial stage of development. In addition, examples of the data include data that is obtained through more specific experiments added as a result of the progress of development and clarification of a treatment mechanism, and that supports treatment efficacy. Further, examples of the data can include data as basic researches at the time when findings different from a hypothesis have occurred, and the like. Specific examples of information about the basic experiments 205 include the dates of experiments, experimenter names, experimenter IDs, experiment names, experiment start times, experiment completion times, IDs of used cells, the names of used cell species, cell counts, and the like. These pieces of information may be included in the quality information.

Types and amounts of data to be acquired can depend on experiment contents. For example, in the case of cell morphology and cell growth evaluation experiments, for example, data to be acquired include cell images, image IDs, image capturing times, culture container Nos., locations in culture containers where images are captured, morphological evaluation results, details at the time of morphological abnormalities, cell counts/cell occupancy rates, and the like. In the case of cell potency evaluation experiments, for example, data to be acquired include culture container Nos., destinations of induction of differentiation, cell images, image IDs, cell morphological evaluation results, flow cytometry measurement results, marker expression evaluation results, and the like. These pieces of information may be included in the quality information.

Data accumulated in the management system 101 is associated with one another first. Data of each step is associated with one another on the basis of a date and/or an operator ID or the like, and data which is consistent lot by lot is created in various types of information about the life cycle from harvesting until transplantation. In particular, the association is important for information having been stored on a system different from the management system 101.

Data formats are changed as necessary. In a case where data having been stored on a system different from the management system 101 is accumulated, the format of the data is modified to be consistent with a format in the management system 101. For example, in a case where different units are used for liquid feed volumes like “10 ml” and “10 cc,” such expressions are modified to be consistent with each other. Basic statistical information is determined for entire data. Maximum/minimum values, averages, variances, standard deviations (SDs), distribution diagrams, and the like are determined for numerical data. Scores are determined for category data, and considered in a similar manner. Regarding methods of determining scores, suitability is evaluated as appropriate.

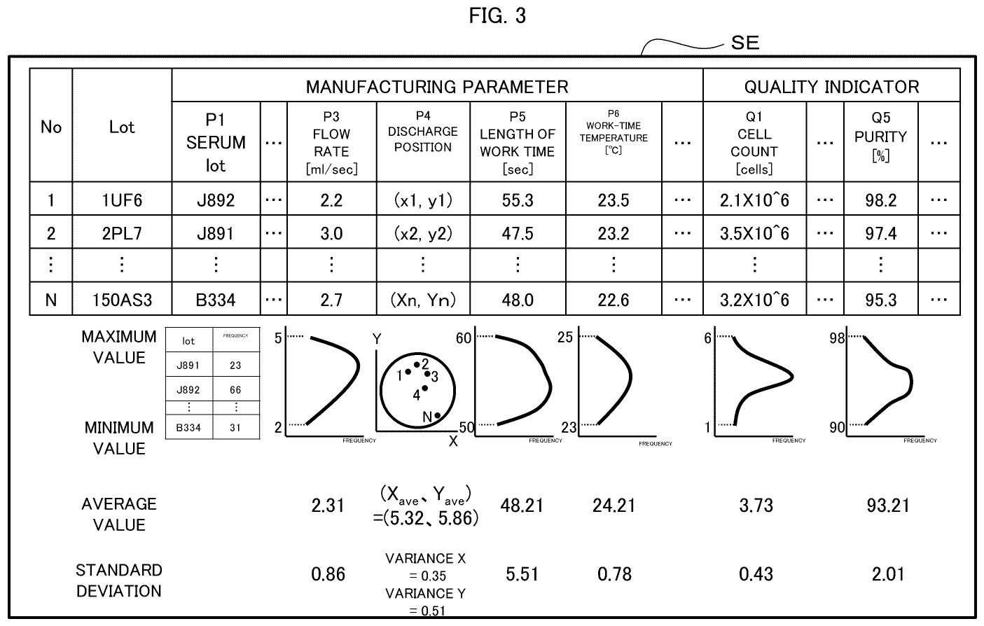

FIG. 3 is an explanatory diagram depicting a screen example SE of the management system 101 according to the first embodiment. The screen example SE in FIG. 3 illustrates a list of statistical information about accumulated data. The screen example SE in FIG. 3 may display various types of information in the life cycle of a cell-processed product or the like, like data related to manufacturing of the cell-processed product or the like accumulated and/or managed by the management system 101, the material management 202 of raw materials and the like, the medical treatment information management 203, the treatment information management 204 of post-transplantation adverse events and/or safety information and the like, or the basic experiments 205.

The screen example SE in FIG. 3 displays both input data whose conditions might be changed, such as parameters at the time of manufacturing, and output data to be analyzed as a result of changes of input conditions, such as post-manufacturing quality or post-transplantation prognostic information. In a case where all pieces of information cannot be displayed on one screen because the information amount is large, the information is divided by information types, timings of occurrence, or the like, and displayed separately. For example, examples of the input data include data related to manufacturing of a cell-processed product or the like, and the material management 202 of raw materials or the like. For example, examples of the input data include serum lots, the flow rates at the time of liquid feeding to culture containers, the discharge positions at the time of liquid feeding to culture containers, the lengths of work time, and the like. For example, examples of the output data include data related to the medical treatment information management 203 described before, and the treatment information management 204 such as post-transplantation adverse events and/or safety information.

In the case of numerical data, the screen example SE in FIG. 3 displays the average, the standard deviation, the maximum value, the minimum value, the distribution diagram, and the like of the numerical data. In the case of category data such as manufacturing locations, serum lots, the names of used apparatuses, the model numbers of used apparatuses, or operator names, the screen example SE in FIG. 3 displays the frequency of occurrence of each category in a table. In the present embodiment, scores are determined by assigning a number to each category as necessary. In the case of numerical data, the screen example SE in FIG. 3 displays the average, the standard deviation, the maximum value, the minimum value, the distribution diagram, and the like of the numerical data. Regarding methods of determining scores, suitability is evaluated as appropriate. In the case of date data, the date data is sorted from the oldest to the latest like category data, and the screen example SE in FIG. 3 displays the frequency of occurrence of each date in a table.

In the case of image data of cells or the like captured at the time of observation with a microscope, the screen example SE in FIG. 3 displays a screen displaying images that are reduced in size such that all the images can be viewed on the one screen, a screen displaying a desired image that is increased in size as necessary, a screen on which, for example, images of the same lot that have been captured on different dates are displayed next to each other, a screen on which images of a plurality of different lots with the same number of culture days are displayed next to each other for comparison, and the like.

In the case of graph data, as with image data, the screen example SE in FIG. 3 displays a screen displaying graphs that are reduced in size such that all the graphs can be viewed on the one screen, a screen displaying a desired graph that is increased in size as necessary, a screen on which, for example, graphs of the same lot that have been captured on different dates are displayed next to each other, a screen on which graphs of a plurality of different lots with the same number of culture days are displayed next to each other for comparison, and the like.

In the case of coordinate data such as the coordinates of a position, in a culture container, of the leading end of a pipette or the like at the time of liquid feeding to the culture container, the screen example SE in FIG. 3 displays such coordinates for each lot in a culture container schematic diagram depicted with XY coordinates. In addition, the screen example SE in FIG. 3 displays the average, the standard deviation, the variance, and the like at each of X and Y coordinates.

In the case of character string data, the screen example SE in FIG. 3 displays entire data in a list. Note that character string data that is used frequently such as “no abnormalities,” “abnormality in appearance,” or “turbidity in medium” has been included as default data in advance in the management system 101. Then, every time such information is input to the management system 101, processing of replacing, with default data, information that can be replaced with the default data is performed, thereby making analysis easy.

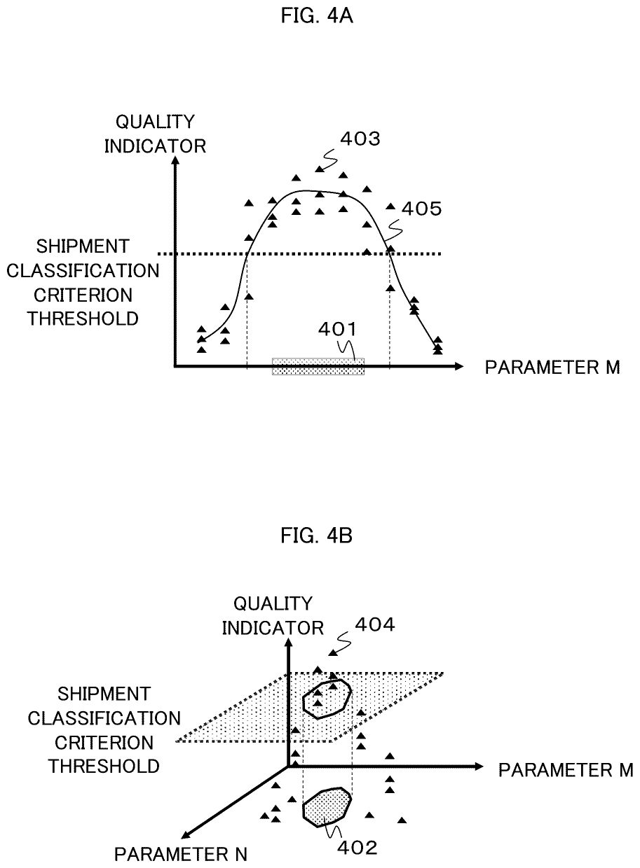

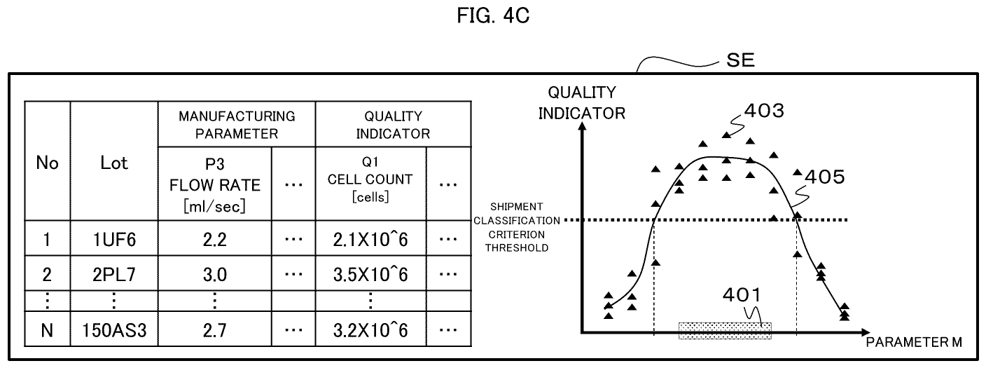

As mentioned before, a manufacturing parameter classification criterion is set on the basis of QbD by using experiment data obtained through experiments (note that a manufacturing parameter classification criterion is a concept encompassing a design space, as mentioned before). In the setting, first, a QTPP of a product is clarified, a QA required for the product is extracted with regard to the QTPP, and a CQA, which is a particularly important quality attribute for quality assurance, is specified. Next, in the setting, a CMA and a CPP are specified from MAs and PPs that influence the CQA. Regarding this process, FIG. 4A depicts an example of a graph of experiment values of each parameter and a quality indicator, and is a graph depicting an example of results obtained by changing one parameter and plotting one quality indicator. FIG. 4B depicts an example of a graph of experiment values of each parameter and a quality indicator, and is a graph depicting an example of results obtained by changing two parameters and plotting one quality indicator.

There are a plurality of parameters and quality indicators, and input variables such as a CQA included in a manufacturing parameter classification criterion are decided as a result of consideration based on QbD. FIG. 4A illustrates a case where a parameter included in a manufacturing parameter classification criterion is a parameter M, and FIG. 4B illustrates a case where parameters included in a manufacturing parameter classification criterion are a parameter M and a parameter N. Note that, for example, the parameters M and N are a flow rate, a work temperature, and the like. Since these are parameters included in the manufacturing parameter classification criteria, FIG. 4A and FIG. 4B also depict manufacturing parameter classification criteria 401 and 402 obtained through experiments in the end. In addition, FIG. 4A and FIG. 4B also depict values 403 and 404 of quality indicators obtained when the parameters are changed. Shipment classification criterion thresholds for deciding whether shipment is allowed or not allowed have been decided in advance for the quality indicators on the basis of experiments, theories, past records, or the like. Note that the shipment classification criterion thresholds can be handled as criterion values in the quality criteria. If the values 403 and 404 of the quality indicators exceed the shipment classification criterion thresholds, shipment is possible, and if the values 403 and 404 of the quality indicators fall below the shipment classification criterion thresholds, the products fail, and shipment is not possible.

In the case of manufacturing with the parameters M and N positioned in the manufacturing parameter classification criteria 401 and 402, the values 403 and 404 of the quality indicators exceed the shipment classification criterion thresholds. Since manufacturing parameter classification criteria may be interpreted as meaning design spaces in the present embodiment, if manufacturing is performed with the parameters M and N in design spaces, the values 403 and 404 of the quality indicators exceed the shipment classification criterion thresholds. In the present embodiment, through a consideration process like this, a manufacturing parameter classification criterion which is an area formed by a multi-dimensional combination of input variables (a CPP, etc.) such as parameters of manufacturing steps and interactions is set. If a cell-processed product manufactured in the end is positioned in the area, it is deemed that the quality is secured.

FIG. 4C is an explanatory diagram depicting a screen example SE of a table (the figure on the left) of each input variable included in a manufacturing parameter classification criterion and values of each quality indicator obtained when parameters are changed, and a graph (the figure on the right) based on the table. Each input variable included in the manufacturing parameter classification criterion and values of each quality indicator obtained when the parameters are changed can be screen-displayed in a table and a graph as in the screen example SE in FIG. 4C. Any combination of a type of input variable included in the manufacturing parameter classification criterion and a type of quality indicator can be screen-displayed by selection by a user. For example, when a parameter and a quality indicator are selected in a screen-displayed table, a graph related to the selected parameter and quality indicator is screen-displayed.

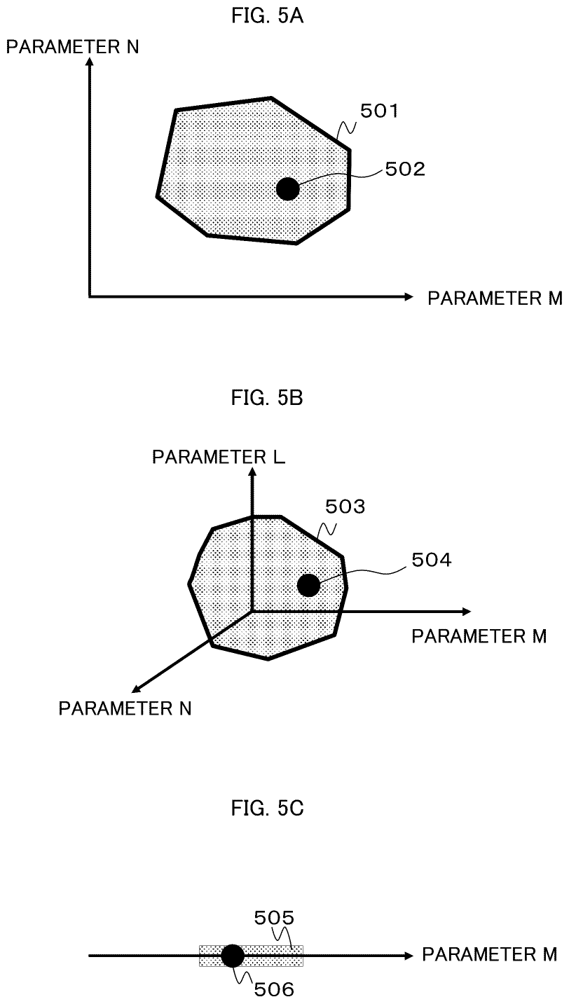

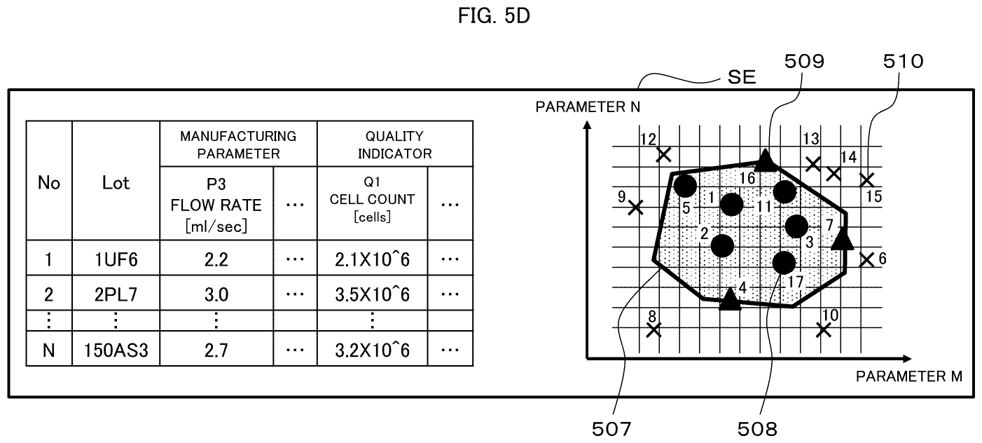

Here, FIG. 5A is an explanatory diagram depicting an example of a two-dimensional manufacturing parameter classification criterion. FIG. 5B is an explanatory diagram depicting an example of a three-dimensional manufacturing parameter classification criterion. FIG. 5C is an explanatory diagram depicting an example of a one-dimensional manufacturing parameter classification criterion.

FIG. 5A depicts a manufacturing parameter classification criterion 501, and a product position 502 representing a cell-processed product plotted as the quality that is attained when the cell-processed product is manufactured with a parameter M and a parameter N. The manufacturing parameter classification criterion 501 is set taking into consideration a multi-dimensional combination of input variables (a CPP, etc.) such as manufacturing steps that have proven to secure quality, and interactions. If a point that is plotted regarding a lot at the time of manufacturing is in the manufacturing parameter classification criterion 501, the lot can be deemed to be able to secure quality. If the point is outside the manufacturing parameter classification criterion 501, the lot can be deemed to be not able to secure quality.

Note that, in FIG. 5A, an area (the mesh portion in FIG. 5A) surrounded by the manufacturing parameter classification criterion 501 is an area that secures quality. In addition, whereas FIG. 5A depicts a two-dimensional criterion including the parameter M and the parameter N, a multi-dimensional criterion is used typically. There are a case where it is deemed that quality can be secured if a product position is in an area surrounded by a manufacturing parameter classification criterion, and a case where a probability that quality is secured is set for each point in an area surrounded by a manufacturing parameter classification criterion. In the latter case, the criterion is a probabilistic manufacturing parameter classification criterion.

As described before, the manufacturing parameter classification criterion 501 is often a multi-dimensional space. Whereas, as an example thereof, FIG. 5A depicts a two-dimensional criterion, as depicted in FIG. 5B, there can be a three-dimensional manufacturing parameter classification criterion 503 including a parameter L, a parameter M, and a parameter N. In this case, too, it can be determined whether or not the quality of a lot can be secured, depending on whether a product position 504 of the lot is inside or outside the manufacturing parameter classification criterion 503. Meanwhile, FIG. 5C depicts a one-dimensional manufacturing parameter classification criterion 505 including a parameter M. In this case, too, it can be determined whether or not the quality of a lot can be secured, depending on whether a product position 506 of the lot is inside or outside the manufacturing parameter classification criterion 505. Note that, instead of using a multi-dimensional manufacturing parameter classification criterion, it is also possible to perform analysis with lowered dimensionality by performing principal component analysis, which is a statistical technique. Further, it is quite possible that the number of parameters, CPPs, or the like that are used to construct a manufacturing parameter classification criterion is greater than three, and in that case, the manufacturing parameter classification criterion is multi-dimensional.

FIG. 5D is an explanatory diagram depicting a screen example SE of a table (left side) of each input variable included in a manufacturing parameter classification criterion and values of each quality indicator obtained when parameters are changed, and a graph (right side) on which lots (Nos. 1 to 17) at the time when each parameter in the manufacturing parameter classification criterion is changed are plotted.

As represented by the screen example SE in FIG. 5D, the manufacturing parameter classification criterion and plotted points that represent lots in a case where respective parameters in the manufacturing parameter classification criterion are changed can be screen-displayed as a graph using symbols such as •, ▴, and x (the lot Nos. 1 to 17 in the figure on the right on the screen example SE in FIG. 5D). Note that •'s are points plotted as quality at the time when manufacturing is performed with certain parameters at a certain time point, and represent that the quality is in a manufacturing parameter classification criterion 507. ▴'s are points plotted as quality at the time when manufacturing is performed with certain parameters at a certain time point, and represent that the quality is on the boundary separating the inside and outside of the manufacturing parameter classification criterion 507. x's are points plotted as quality at the time when manufacturing is performed with certain parameters at a certain time point, and represent that the quality is outside the manufacturing parameter classification criterion 507.

In the first embodiment, as depicted in the screen example SE in FIG. 5D, a table representing parameters and a quality indicator of each lot also can be screen-displayed (the figure on the left on the screen example SE in FIG. 5D). For example, when a user selects a certain lot (a plot 508 (•), a plot 509 (▴), or a plot 510 (x) in the lot Nos. 1 to 17) in the screen-displayed graph, information about the lot in the table can be displayed. In addition, when a user selects a certain lot in the screen-displayed table, the lot (the plot 508, the plot 509, or the plot 510 in the lot Nos. 1 to 17) in the screen-displayed graph can be displayed.

In this manner, a manufacturing parameter classification criterion can be obtained through experiments on the basis of QbD. However, as mentioned before, regenerative medicine uses cells or a biological sample as a raw material, and there are currently many steps to be performed by manual work. Hence, it is difficult to change many conditions, implement experiments a large number of times, and highly accurately obtain reliable data. In a case where less reliable data is obtained, a manufacturing parameter classification criterion determined therefrom is also less reliable. Therefore, in the management system 101 according to the first embodiment, a process in which the influence of each parameter on quality is evaluated by a cell culture simulation taking into consideration cellular fluctuations and a manufacturing parameter classification criterion created on the basis of experiment data is revised is added.

In the first place, each cell population is heterogeneous and is not a uniform group. In addition, each culture environment is not uniform. For example, the inside of an incubator where culture is performed is typically maintained at the temperature of 37° C., but the temperature is not uniform spatially and temporally. For reasons such as the opening and closing of a door of the incubator or the performance of the incubator itself, the inside temperature distribution is not uniform. In addition, cells or a biological sample whose quality is difficult to standardize is used as a raw material, and besides, there are currently many steps to be performed by manual work. Taking into consideration a background like this, cell culture simulation technologies taking into consideration cellular fluctuations have already been published in Reference 1 and Reference 2 (Reference 1: Thi Nhu Trang Nguyen, Kei Sasaki, and Masahiro Kino-Oka. Development of a kinetic model expressing anomalous phenomena in human induced pluripotent stem cell culture. J of Bioscience and Bioengineering. 2021. 131(3). 305-313., Reference 2: Eri Shuzui, Mee-Hae Kim, Keisuke Azuma, Yukako Fujinaga, and Masahiro Kino-Oka. Maintenance of an undifferentiated state of human-induced pluripotent stem cells through botulinum hemagglutinin-mediated regulation of cell behavior. J of Bioscience and Bioengineering. 2019. 127(6). 744-751.).

Cell behavior at a single-cell level such as migration, division, differentiation, or cell death, interactions with surrounding cells such as contact inhibition or inter-cell interactions, behavior and attributes of overall tissues such as collective migration or positional non-uniformity of structures can be computed. In addition, it is possible to cause the influence of culture manipulation performed in manufacturing of a cell-processed product to be reflected. Since a cell population is heterogeneous and a culture environment is not uniform, probabilistic computations are performed as cellular fluctuations. That is, in a cell culture simulation taking into consideration cellular fluctuations, different computation results are output even if the same manufacturing condition is input. The cell culture simulation technologies taking into consideration cellular fluctuations are different from a deterministic simulation technology in which the same computation results are output if the same manufacturing condition is input.

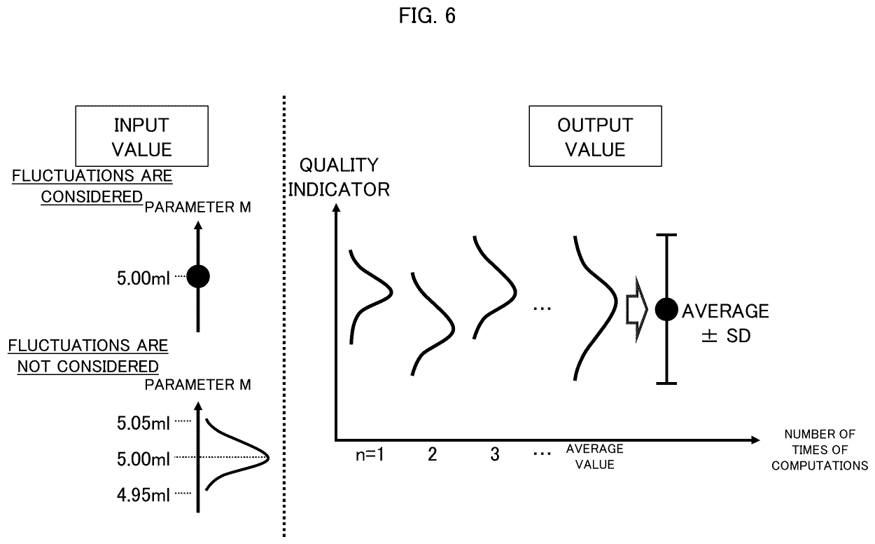

FIG. 6 is an explanatory diagram depicting an example of computation results obtained by performing a cell culture simulation taking into consideration cellular fluctuations. In FIG. 6, computations are performed multiple times using a certain value as an input value regarding one of a plurality of parameters (computations are performed multiple times using 5.00 (ml) as an input value in the example depicted in FIG. 6), and one in a plurality of quality indicators is determined as computations. Note that the manufacturing condition is a combination of the values of respective parameters in a plurality of parameters. In addition, the quality information is a combination of the values of respective quality indicators in a plurality of quality indicators. Since probabilistic computations are performed in a cell culture simulation taking into consideration cellular fluctuations, computation results differ every time a computation is performed. For example, graphs (distributions) obtained in approximate V-shapes differ for each of n=1, 2, 3 . . . . Although the average and the standard deviation (average+SD) of all computation results are determined here as depicted in FIG. 6, other statistical features may be determined. Note that, in a case where cellular fluctuations are not to be taken into consideration, if a certain value (5.00 (ml) in the example depicted in FIG. 6) is input, for example, results of the computations are calculated as normal distributions every time.

The value of a parameter which is an input value has fluctuations in actuality even if one specific value is specified in accordance with a standard operating procedure or the like. For example, in the case of a step to be performed by manual work, the value can vary depending on the proficiency or the like of an operator. In addition, for example, in the case of an automated step, an apparatus itself can generate variations caused by control errors or the like. As an example, in a case where a liquid is fed in a liquid volume of 5 ml, there is a possibility that the actual liquid feed volume varies like 5.00±0.05 ml. Therefore, in computations by a cell culture simulation, variations of an input value may also be taken into consideration. It is possible that the degree of variations of an input value is recognized through experiments, set by a user, and so on. Accordingly, in the present embodiment, preferably, the arithmetic operation section 106 may acquire information (e.g., the value of a probability distribution unique to each parameter such as a liquid feed volume or a flow rate) about the probability distributions of parameters for parameter information (e.g., the value of the liquid feed volume, the value of the flow rate, etc.) input to the input sections 102, 103, and 104, and perform a simulation of a culture process of culture target cells on the basis of the information about the probability distributions of the parameters. By doing so, a more appropriate simulation taking into consideration variations (the probability distributions of the parameters input to the input sections) can be performed.

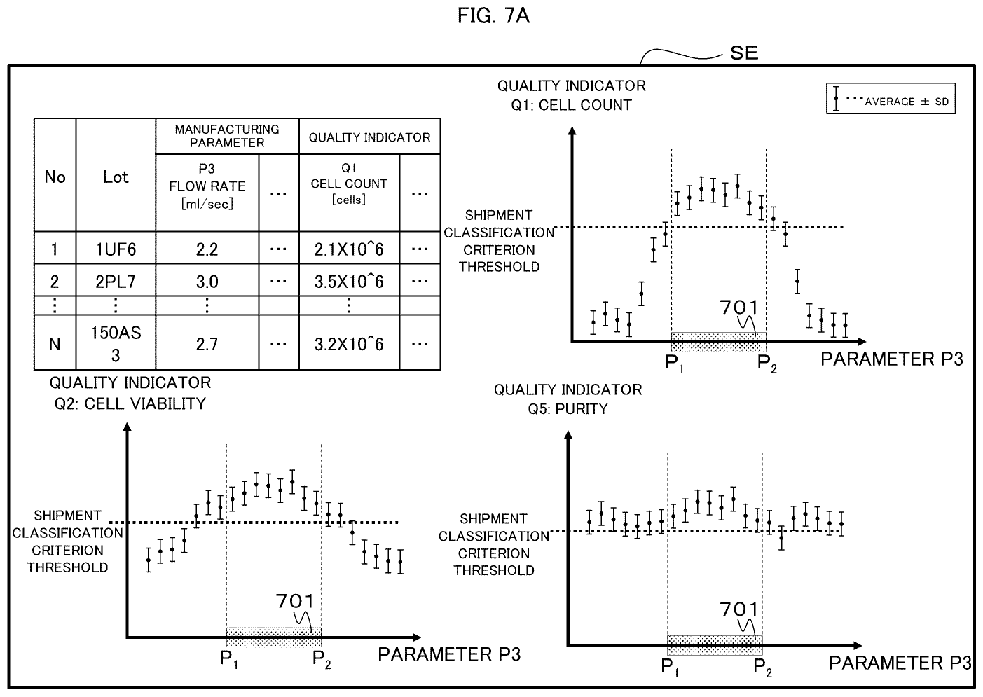

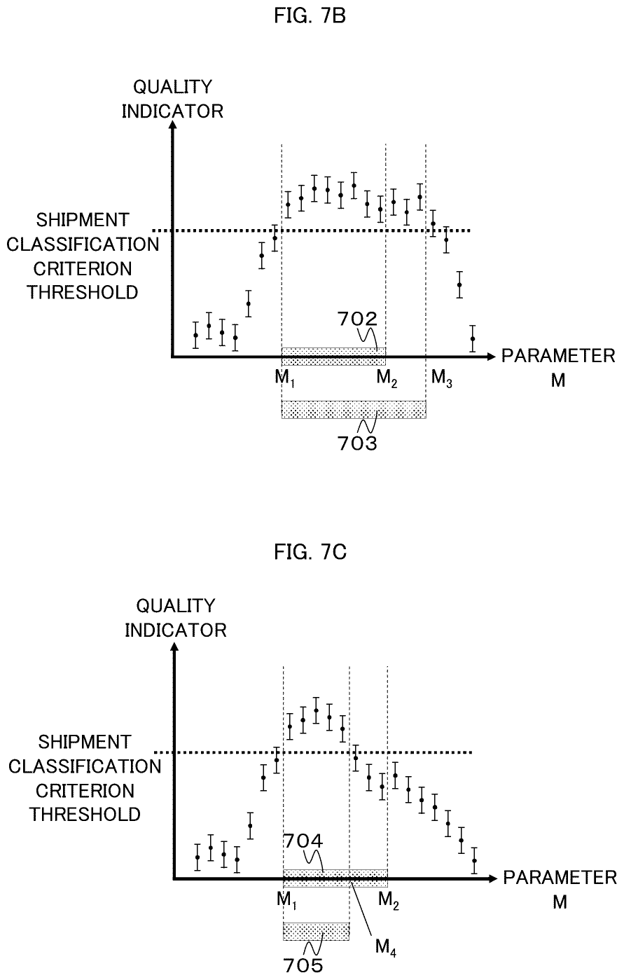

FIG. 7A is an explanatory diagram depicting a screen example SE of a table (the figure on the upper left) depicting computation results obtained by performing a cell culture simulation taking into consideration cellular fluctuations with regard to a manufacturing parameter classification criterion determined through experiments, and graphs (the figure on the upper right, the figure on the lower left, and the figure on the lower right) on which the computation results are plotted. Each graph depicted in FIG. 7A depicts an example of results obtained by changing one parameter and plotting one quality indicator, in the same manner as in FIG. 4A. There are a plurality of parameters and quality indicators like, a flow rate (P3) . . . , a cell count (Q1), cell viability (Q2), purity (Q5) . . . , and the like. An input variable such as a CPP included in a manufacturing parameter classification criterion is decided by performing consideration based on QbD, and FIG. 7A depicts an example in the case of a parameter (flow rate (P3)) included in a manufacturing parameter classification criterion. Since the input variable is a parameter included in the manufacturing parameter classification criterion, FIG. 7A also depicts a manufacturing parameter classification criterion 701 obtained through experiments. With regard to this, FIG. 7A depicts the values of quality indicators obtained when parameters are changed, by using a cell culture simulation taking into consideration cellular fluctuations. Although graphs related to the three quality indicators, namely, the cell count (Q1), the cell viability (Q2), and the purity (Q5), are displayed in FIG. 7A, it is assumed that graphs related to all quality indicators can be displayed by scrolling, switching, or the like of the screen.

P₁ on each of the horizontal axes in FIG. 7A represents one end positioned in the manufacturing parameter classification criterion 701 regarding the parameter P3, and P₂ represents the other end. Values of the quality indicator of each parameter are plotted as average value±standard deviation (average±SD) as explained with reference to FIG. 6. Note that average value±standard deviation may be average value±variance in the present specification and figures. In the example of the case depicted in FIG. 7A, regarding a quality indicator in the case of manufacturing in the range of P₁ to P₂ of the parameter P3 in the manufacturing parameter classification criterion 701, as with results obtained through experiments, results computed in a cell culture simulation taking into consideration cellular fluctuations also exceed a shipment classification criterion threshold. In FIG. 7A, regarding the parameters of the manufacturing parameter classification criterion obtained through experiments, values of the quality indicators are determined by computations using a cell culture simulation taking into consideration cellular fluctuations. As a result, in the case depicted in FIG. 7A, since the computation results also exceed the shipment classification criterion threshold as with experiment results, revision of the manufacturing parameter classification criterion 701 is not necessary.

Note that, although results computed with the parameters included in the manufacturing parameter classification criterion 701 exceed all shipment classification criterion thresholds, results computed with parameters not included in the manufacturing parameter classification criterion 701 do not necessarily fall below all the shipment classification criterion thresholds, and there can be both cases where the results exceed all the shipment classification criterion thresholds and cases where the results fall below all the shipment classification criterion thresholds. These are explained with reference to FIG. 7B and FIG. 7C.

Like FIG. 7A, FIG. 7B is a graph depicting an example of results obtained by changing one parameter and plotting one quality indicator. FIG. 7B depicts an example of the case of a parameter M included in a manufacturing parameter classification criterion. Hence, a manufacturing parameter classification criterion 702 obtained through experiments is also depicted. With regard to this, in the example of the case depicted in FIG. 7B, as a result of plotting values of an quality indicator obtained by changing the parameter M with use of a cell culture simulation taking into consideration cellular fluctuations, values of the quality indicator exceed a shipment classification criterion threshold in a parameter (the range of M₂ to M₃) positioned outside the manufacturing parameter classification criterion 702 which is the range of M₁ to M₂ of the parameter M obtained through experiments. Note that M₁ represents one end of the manufacturing parameter classification criterion 702 obtained through experiments, and M₂ represents the other end. M₃ represents one end of the range where results obtained by determining values of the quality indicator by computations using the cell culture simulation taking into consideration cellular fluctuations exceed the manufacturing parameter classification criterion 702. As depicted in FIG. 7B, unlike experiment results, values of the computation results exceed the manufacturing parameter classification criterion 702 in the range of M₂ to M₃ of the parameter M.

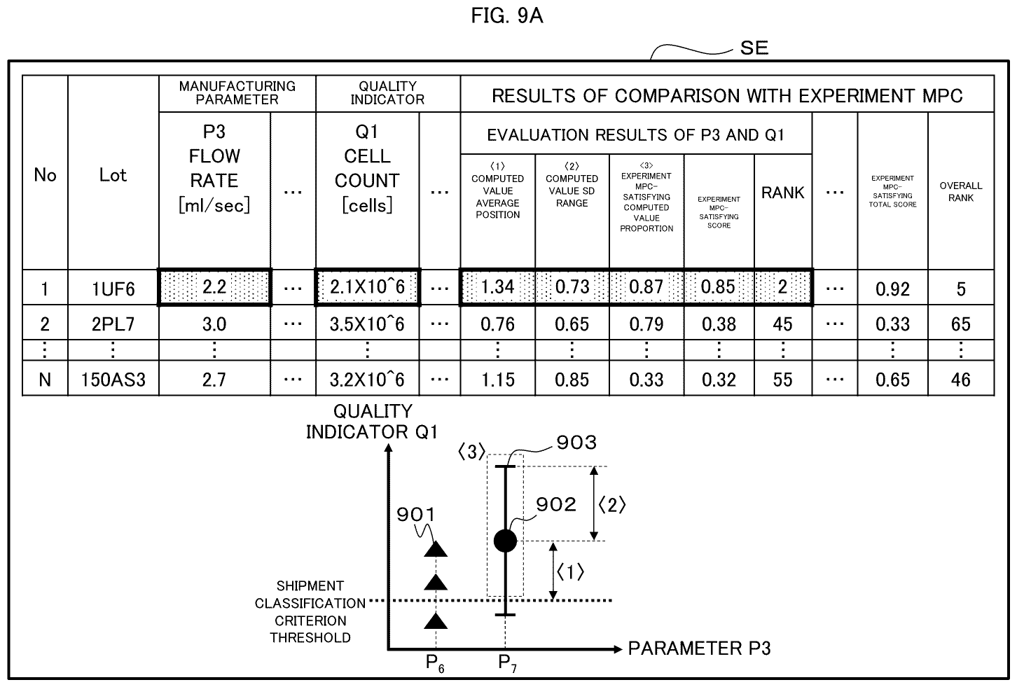

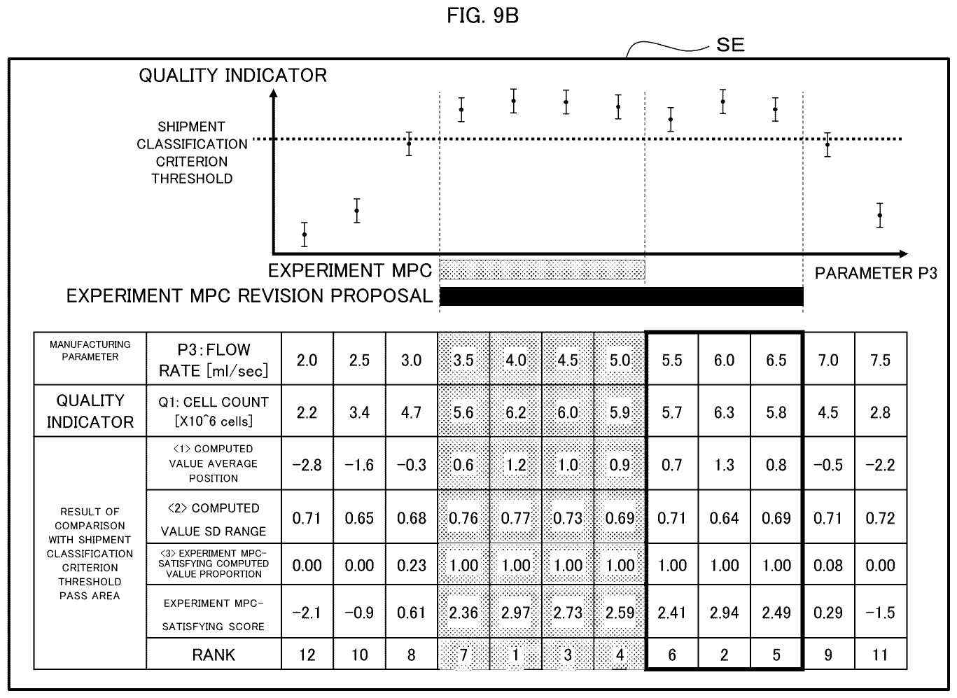

As mentioned before, in constructing a management strategy on the basis of QbD, parameters need to be determined through experiments such that the quality of a cell-processed product is positioned in a manufacturing parameter classification criterion. However, because it is also difficult to secure experiment reproducibility due to the disturbance of cell attributes in experiments using cells, there is a risk that less reliable data is acquired. In a case where a manufacturing parameter classification criterion is determined using such experiment results, there is a possibility that, unlike the experiment results, the parameters also enter the manufacturing parameter classification criterion on the basis of computation results using a cell culture simulation taking into consideration cellular fluctuations. Therefore, the arithmetic operation section 106 performs a cell culture simulation taking into consideration cellular fluctuations and presents a revision proposal 703 (the range of M₁ to M₃ of the parameter M) for the manufacturing parameter classification criterion 702 taking into account results of the simulation. Here, the revision proposal 703 for the manufacturing parameter classification criterion 702 is a range also satisfying a shipment classification criterion threshold of another quality indicator. As depicted in an example of a table in a screen example SE in FIG. 9B mentioned later, as information related to it, the display section 109 such as a screen is caused to output manufacturing stability with regard to shipment classification criterion thresholds of all quality indicators, for example, quantitative information such as scores or ranks, as evaluation indicator information. That is, the arithmetic operation section 106 calculates information about the average value and standard deviation or variance of an quality indicator for each value included in parameter information, and causes the display section 109 such as a screen to output the evaluation indicator information described before, on the basis of the calculated information and information about criterion values (shipment classification criterion thresholds) regarding quality criteria. In addition, the information described before is displayed on the display section 109 such as a screen as visual information representing a state where computation results with regard to a shipment classification criterion threshold of each quality indicator are plotted, as a graph in the screen example SE in FIG. 9B mentioned later. In these manners, it is possible to make it easier for an operator user to perform determination as to whether or not to adopt the revision proposal 703. Explanation is continued with reference back to FIG. 7B. In the first embodiment, a user performs experiments afresh using the revision proposal 703 for the manufacturing parameter classification criterion 702 depicted in FIG. 7B, and considers whether the revision proposal 703 is acceptable or not on the basis of QbD. Note that, in this case, the revision is expansion of the manufacturing parameter classification criterion 702 with use of experiment results. Note that the evaluation indicator information may also include qualitative information, in addition to the quantitative information mentioned before. Examples of the qualitative information include information about cell morphology, information as to whether or not there is turbidity of a medium, information representing a classification result based on such information as to whether or not a shipment classification criterion is satisfied, and the like.

Like FIG. 7A, FIG. 7C is a graph depicting another example of results obtained by changing one parameter and plotting one quality indicator. FIG. 7C also depicts an example of the case of a parameter M included in a manufacturing parameter classification criterion. Hence, a manufacturing parameter classification criterion 704 obtained through experiments is also depicted. With regard to this, in the example of the case depicted in FIG. 7C, as a result of plotting values of an quality indicator obtained by changing the parameter M with use of a cell culture simulation taking into consideration cellular fluctuations, values of the quality indicator fall below a shipment classification criterion threshold in a parameter (the range of M₄ to M₂) positioned inside the manufacturing parameter classification criterion 704 which is the range of M₁ to M₂ of the parameter M obtained through experiments. Note that M₁ and M₂ are similar to those mentioned before. M₄ represents one end of the range where results obtained by determining values of the quality indicator by computations using the cell culture simulation taking into consideration cellular fluctuations fall below the manufacturing parameter classification criterion 704. As depicted in FIG. 7C, unlike experiment results, values of the computation results fall below the manufacturing parameter classification criterion 702 in the range of M₄ to M₂ of the parameter M.

In the mode depicted in FIG. 7C, the arithmetic operation section 106 of the management system 101 performs a cell culture simulation taking into consideration cellular fluctuations and presents a revision proposal 705 (the range of M₁ to M₄ of the parameter M) for the manufacturing parameter classification criterion 704 taking into account results of the simulation. As in the case of FIG. 7B, the revision proposal 705 for the manufacturing parameter classification criterion 704 is a range also satisfying a shipment classification criterion threshold of another quality indicator. As depicted in the example of the table in the screen example SE in FIG. 9B mentioned later, as in the case of FIG. 7B, as information related to it, the display section 109 such as a screen is caused to display manufacturing stability with regard to shipment classification criterion thresholds of all quality indicators, for example, quantitative information such as scores or ranks (evaluation indicator information). In addition, the information described before is displayed on the display section 109 such as a screen as visual information representing a state where computation results with regard to a shipment classification criterion threshold of each quality indicator are plotted, as a graph in the screen example SE in FIG. 9B mentioned later. Explanation is continued with reference back to FIG. 7C. In the first embodiment, a user performs experiments afresh using the revision proposal 705 for the manufacturing parameter classification criterion 704 depicted in FIG. 7C, and considers whether the revision proposal 705 is acceptable or not on the basis of QbD. Note that, in this case, the revision is narrowing of the manufacturing parameter classification criterion 704 with use of experiment results.

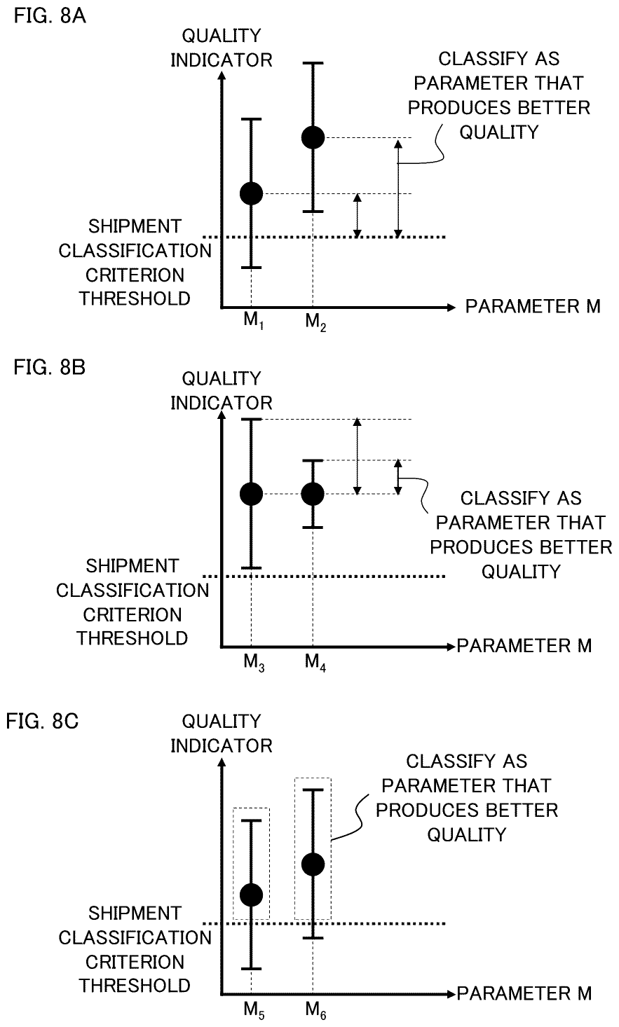

In presenting the revision proposals 703 and 705 for the manufacturing parameter classification criteria 702 and 704, the arithmetic operation section 106 evaluates the degree of stability of computation results in the case of manufacturing with each parameter by using a cell culture simulation taking into consideration cellular fluctuations, with regard to the manufacturing parameter classification criteria 702 and 704 determined through experiments and shipment classification criterion thresholds. All of FIGS. 8A to 8C are explanatory diagrams for explaining indicators for evaluating the degree of stability of computation results in the case of manufacturing with each parameter. Note that average+SD in each of FIGS. 8A to 8C represents a result of simulation about a corresponding one of certain time points M₁ to Me of a parameter M.

With regard to a manufacturing parameter classification criterion determined through experiments and a shipment classification criterion threshold, a possible revision in a case where computation results in the case of manufacturing with each parameter outside the manufacturing parameter classification criterion satisfy the shipment classification criterion threshold is expansion of the manufacturing parameter classification criterion. Computation results in the case of manufacturing with each parameter are results of computations that are performed multiple times, and, in the present embodiment, are expressed by an average value and a standard deviation (or a variance). If all results of computations that are performed multiple times in the case of manufacturing with a certain parameter satisfy a shipment classification criterion threshold, it is considered suitable to make the parameter part of a manufacturing parameter classification criterion. On the other hand, it is considered that, in some cases, some of results of computations that are performed multiple times in the case of manufacturing with a certain parameter satisfy a shipment classification criterion threshold, and the others do not satisfy the shipment classification criterion threshold. It is expected that different institutions that perform regenerative medicine using cell-processed products have different ways of thinking as to ways of thinking about and indicators to be used for determination of the suitability of a parameter as part of a manufacturing parameter classification criterion, which determination is made on the basis of the proportion of computation results satisfying a shipment classification criterion threshold in results of computations that are performed multiple times. In view of this, the degree to which results of computations that are performed multiple times in the case of manufacturing with a certain parameter satisfy a shipment classification criterion threshold is determined as the degree of stability of the computation results in the case of manufacturing with the parameter. Specifically, the relation between results of computations that are performed multiple times in the case of manufacturing with a certain parameter and a shipment classification criterion threshold may be numerically expressed using three indicators depicted in FIGS. 8A to 8C, and, from the three indicators, the stability may be determined as a score representing to what degree the results of computations that are performed multiple times in the case of manufacturing with the parameter satisfy the shipment classification criterion threshold. Further, all parameters may be ranked in order starting from parameters with higher scores representing the degrees to which the shipment classification criterion threshold is satisfied, and ranks of all the parameters are determined.

An indicator depicted in FIG. 8A represents how great the average value (•) of results of computations that are performed multiple times is with regard to a shipment classification criterion threshold. For example, the arithmetic operation section 106 determines the ratio between the average value of the results of computations that are performed multiple times and the shipment classification criterion threshold. As another example, the arithmetic operation section 106 determines the difference between the average value of the results of computations that are performed multiple times and the shipment classification criterion threshold. The greater this value is, the greater the average of the results of computations that are performed multiple times in the case of manufacturing with the parameter is than the shipment classification criterion threshold. That is, the possibility that better quality is attained rises. In the example depicted in FIG. 8A, because the value of the ratio or difference of M₂ is greater than the value of the ratio or difference of M₁, it can be determined that the possibility that better quality is attained is higher in the case of M₂.

An indicator depicted in FIG. 8B represents how small the standard deviation (error bar) or variance of results of computations that are performed multiple times is. For example, the arithmetic operation section 106 determines the reciprocal of the standard deviation (the reciprocal of the variance) of the results of computations that are performed multiple times. The greater this value is, the smaller the standard deviation or variance of the results of computations that are performed multiple times in the case of manufacturing with the parameter is. It can be said that the smaller the standard deviation or variance of the results of computations that are performed multiple times is, the smaller the variations of results of manufacturing is in terms of the quality indicator, and the more stabilized the quality is. In the example depicted in FIG. 8B, because the standard deviation (variance) of M₄ is smaller than the standard deviation (variance) of M₃, it can be determined that the quality is stable.

An indicator depicted in FIG. 8C represents the proportion of computation results satisfying a shipment classification criterion threshold to results of computations that are performed multiple times. The arithmetic operation section 106 sets the upper limit of values of the quality indicator in the results of computations that are performed multiple times in the case of manufacturing with a certain parameter to “average+standard deviation” (or “average+variance”) and sets the lower limit to “average−standard deviation” (or “average−variance”). Then, the proportion of inclusion of an area thereof by the area satisfying the shipment classification criterion threshold is determined. The greater the proportion of inclusion by the area satisfying the shipment classification criterion threshold is, the more frequently the results of computations that are performed multiple times in the case of manufacturing with a certain parameter satisfy the shipment classification criterion threshold. That is, the possibility that better quality is attained rises. In the example depicted in FIG. 8C, because the proportion of inclusion by the area satisfying the shipment classification criterion threshold of M₆ is higher than the proportion of inclusion by the area satisfying the shipment classification criterion threshold of M₅, it can be determined that the possibility that better quality is attained is higher in the case of M₆.

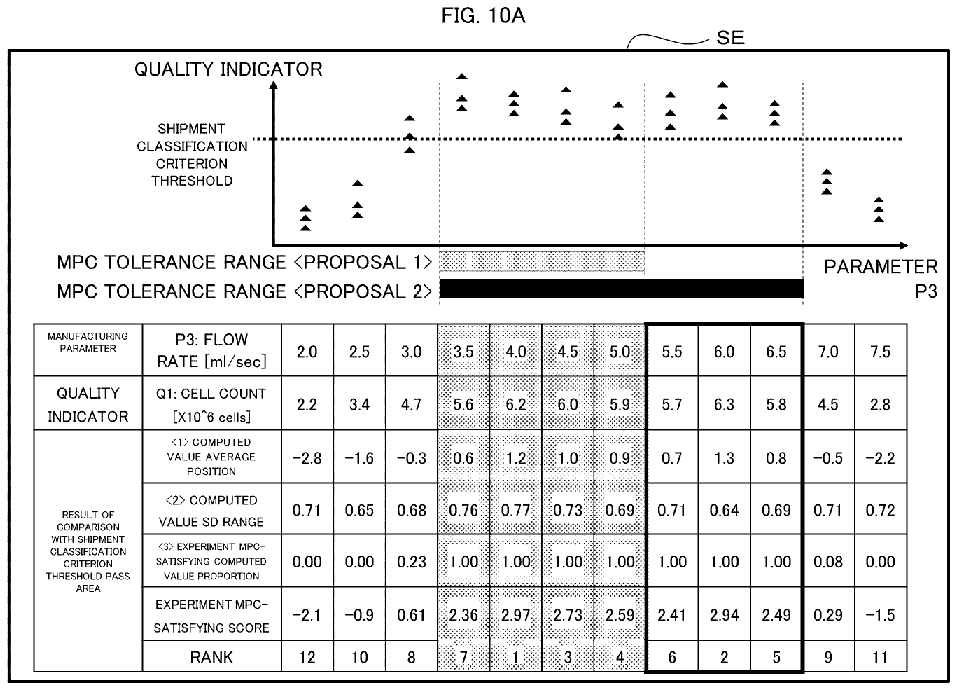

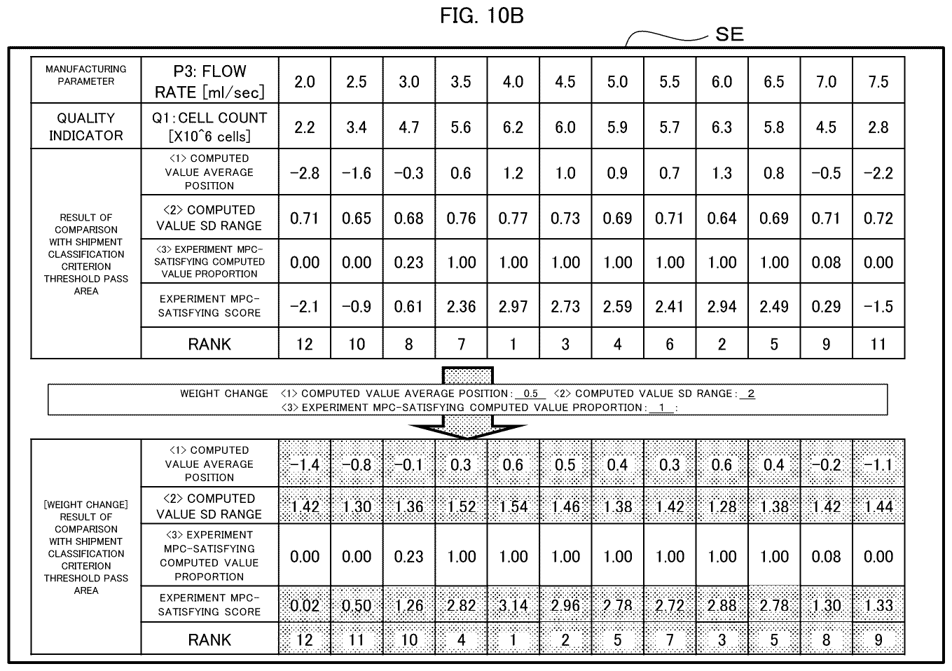

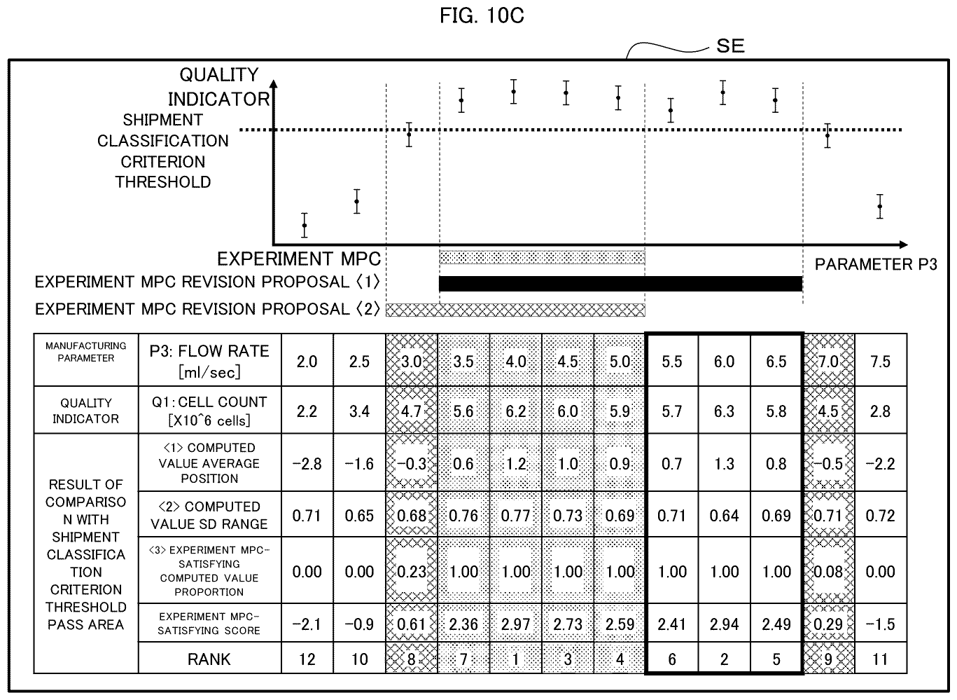

From the three indicators thus determined, a score representing the degree to which results of computations that are performed multiple times in the case of manufacturing with each parameter satisfy a shipment classification criterion threshold is determined. For example, the score may be determined as the product or sum of the three indicators. Further, each indicator may be given a weight depending on determination by a user. These depend on a way of thinking about quality, and it is expected that different institutions that perform regenerative medicine using cell-processed products have different ways of thinking. Since manners of determining scores and manners of giving a weight to each indicator depend on ways of thinking of institutions that perform regenerative medicine, not general users but management users should be allowed to set them depending on the ways of thinking of respective institutions. Details of this are mentioned later with reference to FIG. 10B.

Scores obtained thus far are results of changing values of certain parameters in combinations of the parameters and certain quality indicators. Depending on the combinations of the parameters and the quality indicators, different computation results and evaluation with regard to quality stability are obtained. Basically, only one parameter is changed, and values of one quality indicator are compared. It is also possible to change only the same parameter and compare the values of all quality indicators, but the number of parameters to be changed is desirably one. The reason is because it is difficult to interpret results in a case where a plurality of parameters are changed and values of quality indicators are compared. In some cases, a plurality of parameters are interrelated with each other or are dependent on one another. In that case, a plurality of parameters in such a relation may be changed, and values of a quality indicator may be compared. Analysis may be performed with dimensionality which is lowered by performing a principal component analysis, which is a statistical technique. A total score may be determined using respective scores obtained by various combinations of parameters and quality indicators. For example, the total score may be determined as the product or sum of all scores. In addition, a weight may be applied. A management user is allowed to set methods of computation and whether or not there are weights, depending on the way of thinking of each institution. Further, the arithmetic operation section 106 may rank parameters in order starting from higher total scores, and overall ranks may be determined. Note that the computation amount can become enormous if computations are performed for combinations of all parameters and all quality indicators. Therefore, total scores and overall ranks of parameters which are input variables included in a manufacturing parameter classification criterion may be determined regarding scores and ranks determined about particularly important quality indicators.

Both FIG. 9A and FIG. 9B are explanatory diagrams depicting screen examples SE presenting revision proposal for a manufacturing parameter classification criterion (experiment manufacturing parameter criterion (MPC)) created on the basis of experiment data, by using scores and ranks. Both tables in FIG. 9A and FIG. 9B display together results of each lot determined through experiments and results of computations that are performed multiple times, the results of computations being determined using a cell culture simulation taking into consideration cellular fluctuations.

In the screen example SE in FIG. 9A, some lots in the table are selected, and experiment values 901 related to a parameter that is desired to be evaluated are displayed in a graph (in this figure, three values at the time point of P₆ of the parameter P3). In addition, in the same graph, results of computations that are performed multiple times, the results of computations being determined using a cell culture simulation taking into consideration cellular fluctuations, with regard to the parameter that is desired to be evaluated in the lots selected in the table are displayed by an average value 902 and a standard deviation 903 (in this figure, average+SD at the time point of P₇ of the parameter P3). In the screen example SE in FIG. 9A, values of three stability-related indicators obtained by comparison with the shipment classification criterion threshold mentioned before in the results of computations that are performed multiple times are displayed in the table. The values of the three stability-related indicators are <1> computed value average position, <2> computed value SD range, and <3> experiment MPC-satisfying computed value proportion. When scores and ranks are determined from the values of the three stability-related indicators, the computation may be performed with weights being applied to the indicators. Because the weights depend on ways of thinking about quality, and it is expected that different institutions that perform regenerative medicine using cell-processed products have different ways of thinking, not general users but management users should be allowed to set them depending on the ways of thinking of respective institutions. Details of this are mentioned later with reference to FIG. 10B. In addition, in the screen example SE in FIG. 9A, too, as with what has been described before, total scores and overall ranks can be determined about scores and ranks determined regarding particularly important quality indicators.

In the screen example SE in FIG. 9B, a manufacturing parameter classification criterion (experiment MPC) determined through experiments and results of computations that are performed multiple times, the results of computations being determined using a cell culture simulation taking into consideration cellular fluctuations, are depicted in a graph and a table. The table in the screen example SE in FIG. 9B depicts three indicators <1> to <3>, scores, and ranks about a selected quality indicator with regard to a selected parameter. For example, if values better than the three indicators, the scores, and the ranks of parameters in the manufacturing parameter classification criterion determined through experiments are obtained with a parameter outside the manufacturing parameter classification criterion, a range including the parameter is added to the manufacturing parameter classification criterion determined through experiments. That is, expansion of the manufacturing parameter classification criterion is considered (experiment MPC revision proposal). This expansion is addition of the flow rates 5.5 to 6.5 ml/sec in the table in the screen example SE in FIG. 9B. On the other hand, in a case where the three indicators, the scores, and the ranks of a parameter in the manufacturing parameter classification criterion determined through experiments are clearly inferior to those of other parameters in the manufacturing parameter classification criterion, a range including the parameter is excluded from the manufacturing parameter classification criterion determined through experiments. That is, narrowing of the manufacturing parameter classification criterion is considered (not depicted in the screen example SE in FIG. 9B). Because thresholds of the three indicators, the scores, and the ranks that are used in making proposals to expand and narrow the manufacturing parameter classification criterion depend on ways of thinking about quality, and it is expected that different institutions that perform regenerative medicine using cell-processed products have different ways of thinking, not general users but management users should be allowed to set them depending on the ways of thinking of respective institutions. Details of this are mentioned later with reference to FIG. 10C.

With reference to FIGS. 10A to 10C, functions of the management system 101 used by a management user and screen examples SE thereof are explained. Each of FIGS. 10A to 10C is an explanatory diagram for explaining a screen example SE of the management system 101 used by a management user.

It is expected that different institutions that perform regenerative medicine using cell-processed products have different ways of thinking about quality or the like, and it is assumed that functions related to them and a screen example SE are used by a management user. FIG. 10A depicts a screen example SE of functions of the management system 101 used when a manufacturing parameter classification criterion is determined through experiments. In a method of determining a manufacturing parameter classification criterion on the basis of QbD, experiments are performed on the basis of the design of experiment (DoE) or the like, and a response surface representing the relation of a plurality of CQAs with a CMA or a CPP is created on the basis of a response surface method. The CQAs can include quality indicators. The CMA and the CPP can include parameters. The arithmetic operation section 106 sets a search condition of the CQAs, that is, a target range of quality indicators, and determines a simultaneous optimal solution. Taking into account the obtained simultaneous optimal solution, the arithmetic operation section 106 decides a range of the CMA or the CPP as a manufacturing parameter classification criterion from an overlapping range found by superimposing a tolerated area of the specified CMA or CPP. As depicted in FIG. 10A, in this process, the arithmetic operation section 106 visualizes, using a graph, how obtained experiment data is plotted in each quality indicator when one parameter in the CMA or the CPP is changed in the setting of the search condition of the CQA and the decision of the range of the CMA or the CPP as a manufacturing parameter classification criterion. In addition, the table in the screen example SE in FIG. 10A depicts quantitative information (evaluation indicator information) such as scores or ranks as the degrees of quality stability. In the graph of the screen example SE in FIG. 10A, the X axis is switched depending on selection of the CMA or the CPP. In addition, the Y axis is switched by changing types of quality indicators. Along with this, information about the degrees of quality stability in the table also switches. It is made possible to display the information mentioned before on one screen to represent how each piece of data changes such that pieces of information can be compared with each other in a case where a plurality of ranges are set in the decision of the range of the CMA or the CPP as a manufacturing parameter classification criterion. Using the functions mentioned above, the management system 101 assists determination by a management user in setting of the search condition of the CQA and the decision of the range of the CMA or the CPP as a manufacturing parameter classification criterion. For example, as depicted in the graph in the screen example SE in FIG. 10A, a manufacturing parameter classification criterion (MPC) tolerance range <proposal 1>, an MPC tolerance range <proposal 2>, and the like are screen-displayed, and determination by a management user is assisted.

Next, a screen example SE in which, when scores and ranks are determined from values of three stability-related indicators mentioned with reference to FIG. 9A, a computation is performed with weights being applied to the indicators is explained with reference to FIG. 10B. FIG. 10B depicts a state where a management user inputs weight values to the respective values of the three stability-related indicators on the basis of a way of thinking about quality, and scores and ranks are displayed using the respective values of the three indicators that have changed depending on the weight values. As depicted in FIG. 10B, for example, the weight values of <1> computed value average position, <2> computed value SD range, and <3> experiment MPC-satisfying computed value proportion can be set to 0.5, 2, and 1, respectively. Values after being changed by the weights can be screen-displayed as in the lower table in the screen example SE in FIG. 10B. By referring to the values before and after the changes, appropriate weight values are set.

A screen example SE in which, in revision to expand and narrow the manufacturing parameter classification criterion mentioned with reference to FIG. 9B, a plurality of examples of the range of a manufacturing parameter classification criterion that a management user can tolerate are displayed, and comparison and consideration are performed is explained with reference to FIG. 10C. Regarding selected parameters, three indicators, scores, and ranks depicted in a table in the screen example SE in FIG. 10C are used for a selected quality indicator. For example, if values better than the three indicators, the scores, and the ranks of parameters in a manufacturing parameter classification criterion determined through experiments are obtained with a parameter outside the manufacturing parameter classification criterion, a range including the parameter is added to the manufacturing parameter classification criterion determined through experiments. That is, expansion of the manufacturing parameter classification criterion is considered. Here, what values the three indicators, the scores, and the ranks should take to be determined to be included in the manufacturing parameter classification criterion is based on a way of thinking about quality. On the other hand, in a case where the three indicators, the scores, and the ranks of a parameter in the manufacturing parameter classification criterion determined through experiments are clearly inferior to those of other parameters in the manufacturing parameter classification criterion, a range including the parameter is excluded from the manufacturing parameter classification criterion determined through experiments. That is, narrowing of the manufacturing parameter classification criterion is considered.

It is expected that different institutions that perform regenerative medicine using cell-processed products have different ways of thinking, and it is assumed that functions related to them and a screen example are used by a management user. Therefore, a plurality of examples of the range of a manufacturing parameter classification criterion that the management user can tolerate are displayed on the basis of determination by the management user, and comparison and consideration are performed. In the present example, an experiment MPC whose parameter (P3: flow rate) is 3.5 to 5.0 ml/sec, an experiment MPC revision proposal <1> whose parameter (P3: flow rate) is 3.5 to 6.5 ml/sec, and an experiment MPC revision proposal <2> whose parameter (P3: flow rate) is 3.0 to 5.0 ml/sec are displayed below the graph in the screen example SE in FIG. 10C. Then, in the present example, in the screen example SE in FIG. 10C, computed value average positions of the indicator <1> when the parameter (P3: flow rate) is 3.0 ml/sec and 7.0 ml/sec are −0.3 and −0.5, which are near and slightly below a shipment classification criterion threshold. Accordingly, in the present example, the management user selects the experiment MPC revision proposal <1> in which the manufacturing parameter classification criterion does not include these parameters, and causes the output section 108 to display the experiment MPC revision proposal <1>. Note that, although a table, a graph, and a revision proposal related to narrowing are not depicted in the screen example SE in FIG. 10C, for example, it is possible that the arithmetic operation section 106 selects the range of 4.0 to 5.0 ml/sec of the parameter (P3: flow rate) as a revision proposal of narrowing and causes the output section 108 to display the revision proposal. In this case, importance can be placed on stability more than in the case of the experiment MPC.

### <Management Method>

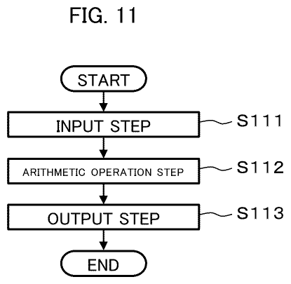

A management method in which the management system 101 having the functions mentioned above is used and a manufacturing parameter classification criterion created on the basis of experiment data is revised is explained. FIG. 11 is a flowchart for explaining contents of a management method according to the first embodiment.

As depicted in FIG. 11, the management method has an input step S111, an arithmetic operation step S112, and an output step S113.

At the input step S111, parameter information related to parameters of processing of culture target cells by at least culture is input.

At the arithmetic operation step S112, on the basis of the parameter information input at the input step S111 and cell attribute information which is information about the probability distribution of cell attributes of the culture target cells, a simulation of a culture process of the culture target cells is performed.

At the output step S113, information about a first manufacturing parameter classification criterion is output, the information about the first manufacturing parameter classification criterion being information based on experiment data of an experiment of culturing the culture target cells and information about a quality criterion of a product related to the culture target cells, and being information about a manufacturing condition that should be satisfied when the culture target cells are cultured.

Here, in the management method according to the first embodiment, at the arithmetic operation step S112, on the basis of results of the simulation described before and the information about the quality criterion, for each of values included in the parameter information, evaluation indicator information which is an evaluation indicator with regard to a quality criterion of a quality indicator at the time when the culture target cells are cultured using the value is calculated, and information about a revision proposal for the first manufacturing parameter classification criterion based on the evaluation indicator information is caused to be output at the output step S113.

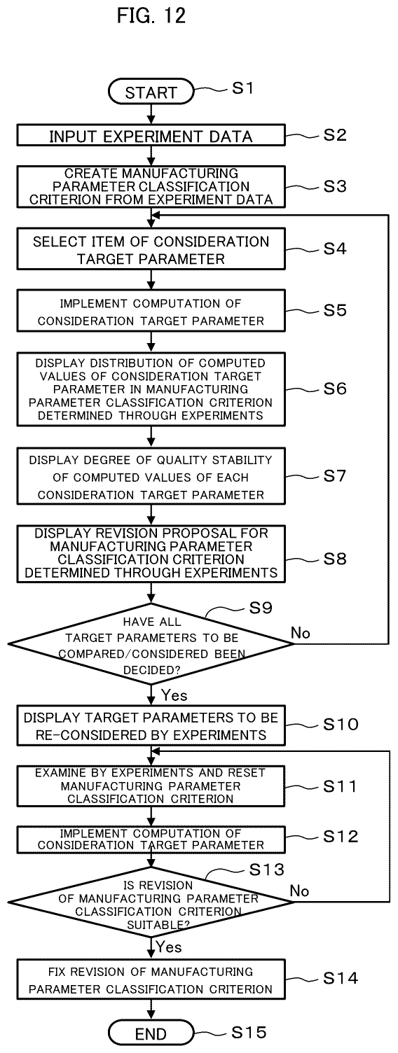

FIG. 12 is a flowchart for explaining a series of more specific procedures of the management method according to the first embodiment explained above. As depicted in FIG. 12, the management method has Steps S1 to S15. Note that the input step S111 described before is equivalent to Step S2. The arithmetic operation step S112 described before is performed at Steps S3 to S14. The output step S113 described before is performed when displaying is performed at Steps S6 to S8, S10, and the like. Hereinafter, Steps S1 to S15 in the management method are explained.

### <Step S1: Start>

The management system 101 is activated.

### <Step S2: Input Experiment Data>

All pieces of information in the life cycle of a cell-processed product or the like, like information about respective steps such as harvesting, purification, gene transfer, culture, condensation, transportation, or transplantation, information about material management of raw materials and the like, and medical treatment information such as post-transplantation adverse event and/or safety information, are input to the management system 101 through the input sections 102, 103, and 104. The input method is selected on the basis of the mode of input-source data as depicted in FIG. 1 and FIG. 2A.

After the input, features of various types of information are displayed on the display section 109 as depicted in the screen example SE in FIG. 3. For example, in the case of numerical data, the average, the standard deviation, the maximum value, the minimum value, the distribution diagram, and the like of the numerical data are displayed. In the case of category data, the frequency of occurrence of each category is displayed in a table. In the case of character string data, depending on contents thereof, analysis is made easy by performing, for example, a process of replacement with data having been put in as default data in advance in the management system 101. In addition, various types of output data such as post-manufacturing quality or post-transplantation prognostic information to be analyzed as a result of changes of input conditions are displayed.

<Step S3: Create Manufacturing Parameter Classification Criterion from Experiment Data>

On the basis of QbD, a manufacturing parameter classification criterion which is an area formed by a multi-dimensional combination of input variables (a CPP, etc.) such as parameters of manufacturing steps and interactions is set.

### <Step S4: Select Item of Consideration Target Parameter>

A user selects a consideration target parameter. Basically, an input variable (a CPP, etc.) such as a parameter of a manufacturing step included in the manufacturing parameter classification criterion is mainly selected.

### <Step S5: Implement Computation of Consideration Target Parameter>

Using a cell culture simulation taking into consideration cellular fluctuations, the user determines values of quality indicators by computations with regard to the consideration target parameter selected at Step S4. Since there are a plurality of quality indicators, computations are performed for all the quality indicators.

### <Step S6: Display Distribution of Computed Values of Consideration Target Parameter in Manufacturing Parameter Classification Criterion Determined Through Experiments>

As depicted in the screen example SE in FIG. 7A, computation results are output on the display section 109 of the management system 101, for example, on a screen. For example, the values of the plurality of quality indicators determined through computations are expressed as “average value±standard deviation” (average±SD). In particular, it is represented whether or not a computation result satisfies a shipment classification criterion threshold in classifying a case where a parameter is inside the manufacturing parameter classification criterion and a case where a parameter is outside the manufacturing parameter classification criterion.

### <Step S7: Display Degree of Quality Stability of Computed Values of Each Consideration Target Parameter>

As depicted in FIGS. 8A to 8C, three indicators, namely, the average value of results of computations that are performed multiple times, the standard deviation of the results of computations that are performed multiple times, and the proportion of computation results included in a quality pass area (an area above the shipment classification criterion threshold) specified by the shipment classification criterion threshold when the upper limit of values of the quality indicator is “average+standard deviation” and the lower limit is “average−standard deviation,” a score expressing the quality stability determined from the three indicators, and a rank in descending order of score are determined as the degree of quality stability with regard to the shipment classification criterion threshold, and are output to the display section 109 of the management system 101.

### <Step S8: Display Revision Proposal for Manufacturing Parameter Classification Criterion Determined Through Experiments>

As depicted in FIG. 9B, in classifying a case where a parameter is inside the manufacturing parameter classification criterion and a case where a parameter is outside the manufacturing parameter classification criterion, taking into account the degree of quality stability quantitatively represented at Step S7 as to whether a computation result satisfies the shipment classification criterion threshold, a revision proposal for the manufacturing parameter classification criterion determined through experiments is output to the display section 109 of the management system 101.

<Step S9: Have all Target Parameters to be Compared and Considered been Decided?>

After Step S8, in a case where all consideration target parameters (target parameters to be compared and considered) that are considered to be necessary for re-consideration of the manufacturing parameter classification criterion determined through experiments have been decided (Yes at Step S9), the procedure proceeds to Step S10. In a case where all consideration target parameters have not been decided (No at Step S9), the procedure returns to Step S4, and consideration is performed again. It is sufficient if whether or not all consideration target parameters have been decided is basically decided about input variables (a CPP, etc.) such as parameters in manufacturing steps included in a manufacturing parameter classification criterion, but parameters that are deemed to influence the quality may be added as appropriate.

### <Step S10: Display Target Parameters to be Re-Considered Through Experiments>

Consideration target parameters that are considered to be necessary for re-consideration of the manufacturing parameter classification criterion which is output by the management system 101 and is determined through experiments are displayed on the display section 109.

### <Step S11: Examine by Experiments and Reset Manufacturing Parameter Classification Criterion>

The user performs experiments again handling a displayed consideration target parameter as a target and resets a manufacturing parameter classification criterion on the basis of QbD again.

### <Step S12: Implement Computation of Consideration Target Parameter>

Using a cell culture simulation taking into consideration cellular fluctuations, as in Step S5, the user determines values of quality indicators by computations with regard to the consideration target parameter. Since there are a plurality of quality indicators, computations are performed for all the quality indicators.

### <Step S13: Is Revision of Manufacturing Parameter Classification Criterion Suitable?>

After Step S12, it is evaluated whether consistency is attained between re-consideration results of the manufacturing parameter classification criterion determined through experiments and computation results obtained by performing a cell culture simulation taking into consideration cellular fluctuations. In a case where consistency is not attained (No at Step S13), the procedure returns to Step S11. In a case where consistency is attained (Yes at Step S13), the procedure proceeds to Step S14.

### <Step S14: Fix Revision of Manufacturing Parameter Classification Criterion>

In a case where consistency is attained between the re-consideration results of the manufacturing parameter classification criterion determined through experiments and the computation results obtained by performing the cell culture simulation taking into consideration cellular fluctuations (Yes at Step S13), the revised manufacturing parameter classification criterion is fixed.

### <Step S15: End>

After the consideration ends, consideration results are electronically stored on the auxiliary storage section 107 in the storage. The user ends the operation of the management system 101 by appropriate operation.

The management system 101 and the management method according to the first embodiment explained above can evaluate the influence of each parameter on the quality by a cell culture simulation taking into consideration cellular fluctuations and revise a manufacturing parameter classification criterion (first manufacturing parameter classification criterion) created on the basis of experiment data. The management system 101 and the management method implement computations multiple times under all manufacturing conditions in the cell culture simulation and output quality indicators reflecting the disturbance of cell attributes. The management system 101 and the management method numerically express the proportion or the like of inclusion of each manufacturing condition by a manufacturing parameter classification criterion (design space) created on the basis of experiment data. The management system 101 and the management method evaluate the distribution of results of the cell culture simulation with regard to the manufacturing parameter classification criterion created on the basis of experiment data and determine whether or not a revision such as expansion or narrowing of the manufacturing parameter classification criterion is necessary. In this manner, the management system 101 and the management method can enhance the precision of a manufacturing parameter classification criterion by evaluating a combination of experiment data and computation data, and as a result, realize quality stabilization of a cell-processed product.

### Second Embodiment

The management system 101 according to a second embodiment is explained.

In the second embodiment, first, data related to various types of information in the life cycle of a cell-processed product or the like, the data having been obtained until a certain time point, is input as quality information to the management system 101. Examples of the data include information about respective steps related to the manufacturing 201 such as harvesting, purification, gene transfer, culture, condensation, formulation, transportation, or transplantation, information about the material management 202 of raw materials and the like, and information about the medical treatment information management 203 such as post-transplantation adverse events and/or safety information. The quality information is input through the input sections 102, 103, and 104.

In manufacturing at and after the certain time point, data having been obtained until an intermediate step of manufacturing (at least one of manufacturing information, treatment information, and transportation information about a cell-processed product being manufactured) is input to the management system 101. The data is also input through the input sections 102, 103, and 104. The arithmetic operation section 106 computes where the data having been obtained up to that time point is positioned in a manufacturing parameter classification criterion, and causes the display section 109 to display a result of the computation. Simultaneously, using a cell culture simulation taking into consideration cellular fluctuations, the arithmetic operation section 106 determines a computation result related to quality in the case of manufacturing with parameters having been used until the intermediate step of manufacturing (the current quality condition is calculated). Using the computation results, for example, as the degree of product stability, three indicators, scores, and ranks of selected parameters are evaluated with regard to a selected quality indicator.

Using them, the arithmetic operation section 106 predicts the quality at the end of manufacturing from the data having been obtained until the intermediate step of manufacturing. As in the first embodiment, the quality criterion to be used is a quality criterion at the time of completion of a cell-processed product. On the basis of the manufacturing parameter classification criterion and quality information about each cell-processed product at the intermediate step of manufacturing, the arithmetic operation section 106 predicts whether each cell-processed product passes or fails at the time of completion of the cell-processed product (i.e., predicts whether the cell-processed product passes or fails a quality criterion at the time of completion).

A specific technique for predicting data at the time of completion (which may be quality information or may be a product position in the manufacturing parameter classification criterion) on the basis of quality information at an intermediate step of manufacturing is a design that can be determined as appropriate by those skilled in the art. For example, a function that accepts, as an input, quality information at an intermediate step of manufacturing and outputs a product position may be pre-stored. Specific contents of the function can be defined as appropriate by those skilled in the art on the basis of known technologies or the like. In addition, machine learning can also be used.

In a case where it is found that data having been obtained until an intermediate step of manufacturing is positioned outside a manufacturing parameter classification criterion, a user may choose to stop manufacturing of the product or the management system 101 may automatically adopt the choice. As compared to a case where the quality is evaluated at the end of manufacturing and it is found that a shipment classification criterion is not satisfied, cost can be reduced by predicting that a shipment classification criterion is not satisfied and stopping manufacturing at an intermediate step of manufacturing.

In addition, it is also possible that, by changing the manufacturing method in a case where it is predicted at an intermediate step of manufacturing that a shipment classification criterion is not to be satisfied, it is deemed that the shipment classification criterion is to be satisfied at the end of manufacturing. It should be noted that, in that case, it is preferable if, when a manufacturing license is obtained, the manufacturing method is permitted to be changed during manufacturing.

As another example, a threshold for determining whether an intermediate product at an intermediate step of manufacturing is allowed to move onto the subsequent steps, that is, a manufacturing parameter classification criterion for the intermediate product, may be provided, and evaluation may be performed with regard to the manufacturing parameter classification criterion. In addition, as in the first embodiment, it is also possible that a manufacturing parameter classification criterion for an intermediate product determined through experiments in advance is evaluated on the basis of computation results obtained by performing a cell culture simulation taking into consideration cellular fluctuations, and a revision proposal for the manufacturing parameter classification criterion for the intermediate product is output. That is, it is also possible that the arithmetic operation section 106 calculates at least one of a quality condition with regard to a quality criterion of an intermediate product of a cell-processed product being manufactured and a quality condition with regard to a quality criterion of the cell-processed product at the end of each step, and causes the display section 109 to output a revision proposal for a manufacturing parameter classification criterion with regard to the quality condition of the cell-processed product. Then, it is also possible that the revision proposal is considered through experiments. By doing so, a revision proposal for a manufacturing parameter classification criterion can be checked and considered at an intermediate step of manufacturing, and hence, quality stabilization of a cell-processed product can be realized more appropriately. In addition, by checking and considering the revision proposal at the intermediate step of manufacturing, the procedure can be prevented from proceeding to the subsequent steps in a case where it is determined that the shipment classification criterion is not satisfied, and accordingly, cost can be reduced.

### Third Embodiment

The management system 101 according to a third embodiment is explained. In the third embodiment, data obtained through experiments and computation results obtained by performing a cell culture simulation taking into consideration cellular fluctuations are input to the management system 101 and are handled equivalently. That is, the arithmetic operation section 106 adds the computation results obtained by performing the simulation of a culture process of culture target cells to the experiment data and performs a simulation further. Then, setting of a manufacturing parameter classification criterion and the like are performed on the basis of QbD.

Experiments in regenerative medicine require cost and time, and it is also difficult to secure experiment reproducibility due to the disturbance of cell attributes. It is thus not easy to increase the number of times of experiments and obtain reliable data highly accurately. Therefore, by adding computation results obtained by performing a cell culture simulation taking into consideration cellular fluctuations to data obtained through experiments, the management system 101 increases the number of pieces of data and enhances the quality of the data.

Note that data obtained through experiments and data obtained through computations can also be handled with changed weights by setting weight values. If data obtained through experiments is results obtained actually using cells and higher importance is placed on the data than on data obtained through computations, the weight value of the data obtained through computations is set lower than one. Meanwhile, if it is assumed that data obtained through experiments can vary depending on operators and higher importance is placed on the fact that data obtained through computations can be tested an increased number of times, the weight value of the data obtained through computations is set higher than one.

### Fourth Embodiment

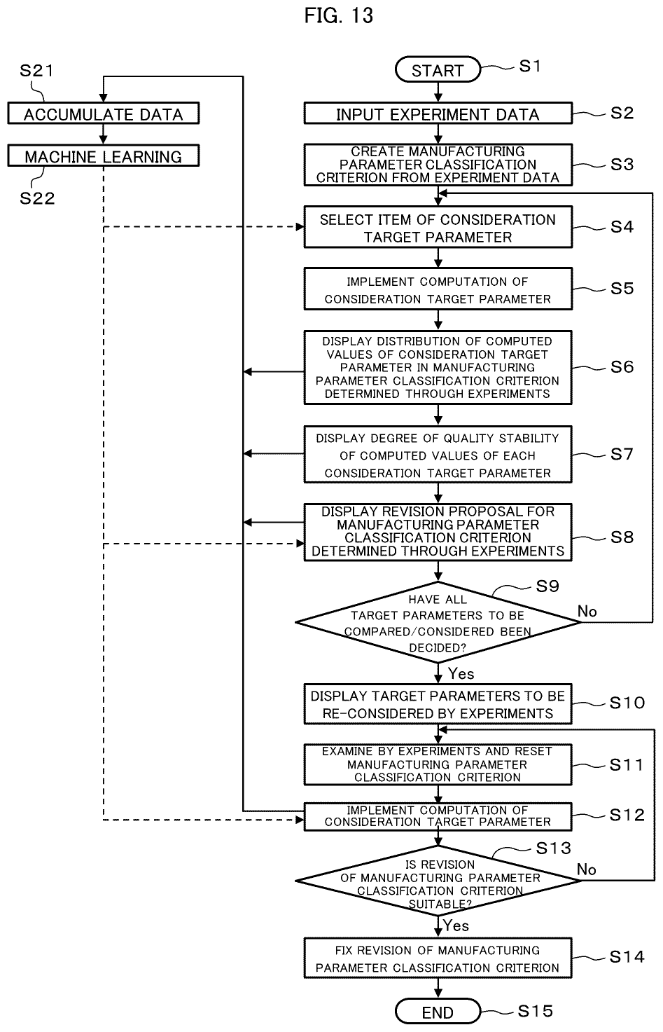

A management method according to a fourth embodiment is explained. FIG. 13 is a flowchart for explaining a series of specific procedures of the management method according to the fourth embodiment. As can be known by comparison with the flowchart in FIG. 12, the management method according to the fourth embodiment is different from the management method according to the first embodiment in that the management method according to the fourth embodiment has Step S21 and Step S22. The following mainly explains the difference.

In the management method according to the present fourth embodiment, a revision proposal for a manufacturing parameter classification criterion determined through experiments is presented using machine learning.

As depicted in FIG. 13, in the management method according to the fourth embodiment, results of operations performed by a user at the arithmetic operation step S112, specifically, Steps S6, S7, S8, and S12 described before, are accumulated, and, using them, a machine learning model that selects parameters and the like and presents revision proposals for a manufacturing parameter classification criterion (first manufacturing parameter classification criterion) at Steps S4, S8, S12, and the like is generated.

Specifically, as depicted in FIG. 13, data is accumulated as appropriate at Step S21, machine learning is performed at Step S22, and a result thereof is reflected at Steps S4, S8, S12, and the like.

Since it is sufficient if well-known or known techniques such as neural network or logistic regression are adopted for the machine learning model, details are not mentioned in the present embodiment. In selection at Steps S4, S8, S12, and the like performed by a user, selection by machine learning is also displayed.

In machine learning, it is possible to output a parameter range and a manufacturing parameter classification criterion having been changed with the highest change priority, using, as inputs, a parameter range and a manufacturing parameter classification criterion yet to be changed. In addition, for example, in machine learning, it is possible to create teaching data that uses, as inputs, a parameter range and a manufacturing parameter classification criterion yet to be changed and uses, as outputs, a parameter range and a manufacturing parameter classification criterion with the highest change priority, at Step S8 and/or S12.

By using the trained model thus created, a preferable parameter range having been changed can be calculated on the basis of a parameter range yet to be changed, at Steps S4, S8, S12, and the like. The calculated range may be reflected as a range with the highest change priority at Step S12. Change recommendation information, that is, a revision proposal, is output in this manner.

Accordingly, the management method according to the present fourth embodiment can enhance the precision of consideration contents, and as a result, realize quality stabilization of a cell-processed product or the like. Furthermore, product manufacturing taking into consideration an enormous number of parameters related to a cell-processed product can be realized.

What is claimed is:

1. A management system comprising:
a storage section that stores cell attribute information which is information about a probability distribution of a cell attribute of culture target cells, experiment data of an experiment of culturing the culture target cells, and information about a quality criterion of a product related to the culture target cells;
an input section to which parameter information is input, the parameter information being related to a parameter of processing of the culture target cells by at least culture;
an arithmetic operation section that performs a simulation of a culture process of the culture target cells on a basis of the parameter information input to the input section and the cell attribute information; and
an output section that outputs information about a first manufacturing parameter classification criterion on a basis of the experiment data and the information about the quality criterion, the information about the first manufacturing parameter classification criterion being information about a manufacturing condition that should be satisfied when the culture target cells are cultured, wherein
the arithmetic operation section is configured to calculate evaluation indicator information on a basis of a result of the simulation and the information about the quality criterion, the evaluation indicator information being an evaluation indicator with regard to the quality criterion of a quality indicator at time when the culture target cells are cultured using each of values included in the parameter information, and cause information about a revision proposal for the first manufacturing parameter classification criterion based on the evaluation indicator information to be output.

2. The management system according to claim 1, wherein the arithmetic operation section is configured to calculate information about an average value and a standard deviation or a variance of the quality indicator for each of the values included in the parameter information, and cause the evaluation indicator information to be output on a basis of the calculated information and information about a criterion value of the quality criterion.

3. The management system according to claim 1, wherein the arithmetic operation section is configured to acquire information about a probability distribution of the parameter for the parameter information input to the input section, and perform the simulation of the culture process of the culture target cells on a basis of the information about the probability distribution of the parameter.

4. The management system according to claim 1, wherein the arithmetic operation section is configured to accept, as an input, at least one of manufacturing information, treatment information, and transportation information about a cell-processed product being manufactured, calculate a current quality condition by using the input, and predict a pass or a fail with regard to a quality criterion of the cell-processed product at time of completion.

5. The management system according to claim 1, wherein the arithmetic operation section is configured to calculate at least one of a quality condition with regard to a quality criterion of an intermediate product of a cell-processed product being manufactured and a quality condition with regard to a quality criterion of the cell-processed product at an end of each step, and cause the output section to output a revision proposal for a manufacturing parameter classification criterion with regard to the quality condition of the cell-processed product.

6. The management system according to claim 1, wherein the arithmetic operation section is configured to add a computation result obtained by performing the simulation of the culture process of the culture target cells to the experiment data, and further perform a simulation.

7. A management method comprising:
an input step at which parameter information is input, the parameter information being related to a parameter of processing of culture target cells by at least culture;
an arithmetic operation step at which a simulation of a culture process of the culture target cells is performed on a basis of the parameter information input at the input step and cell attribute information which is information about a probability distribution of a cell attribute of the culture target cells; and
an output step at which information about a first manufacturing parameter classification criterion is output, the information about the first manufacturing parameter classification criterion being information based on experiment data of an experiment of culturing the culture target cells and information about a quality criterion of a product related to the culture target cells, and being information about a manufacturing condition that should be satisfied when the culture target cells are cultured, wherein,
at the arithmetic operation step, evaluation indicator information is calculated on a basis of a result of the simulation and the information about the quality criterion, the evaluation indicator information being an evaluation indicator with regard to the quality criterion of a quality indicator at time when the culture target cells are cultured using each of values included in the parameter information, and, at the output step, information about a revision proposal for the first manufacturing parameter classification criterion based on the evaluation indicator information is output.

8. The management method according to claim 7, wherein a machine learning model is generated at the arithmetic operation step, the machine learning model being configured to present a revision proposal for the first manufacturing parameter classification criterion by using a result obtained through the simulation.
